(19) United States
(12) Patent Application Publication
(10) Pub. No.: US 2012/0156281 A1
(43) Pub. Date: Jun. 21, 2012

(54) Meningococcal Vaccine Based on Lipooligosaccharide (LOS) and Neisseria Meningitidis Protein

(75) Inventors: Jean Haensler, Grezieu La Varenne (FR); Bruno Guy, Lyon (FR); Noëlle Mistretta, Saint Bel (FR); Monique Moreau, Lyon (FR); Geneviève Renauld-Mongenie, Chaponost (FR); Bachra Rokbi, Lyon (FR)

(73) Assignee: SANOFI PASTEUR, Lyon cedex 07 (FR)

(21) Appl. No.: 13/319,868

(22) PCT Filed: May 12, 2010
(86) PCT No.: PCT/FR2010/000368

§ 371 Date: Jan. 27, 2012

(30) Foreign Application Priority Data
May 14, 2009 (FR) 0902330
May 14, 2009 (FR) 0902333

(51) Int. Cl. A61K 39/095; A61P 37/04; A61P 31/04; A61K 9/127

(52) U.S. Cl. 424/450; 424/250.1

(57) ABSTRACT

The invention relates to a vaccine against N. meningitidis infections, comprising (i) an N. meningitidis LOS especially consisting of a lipid A, an inner core, and an L8-type α chain in which the heptose II residue of the inner core (a) carries a phosphoethanolamine (PEA) substituent in position O-3, and does not carry a PEA substituent in positions O-6 and O-7, or (b) carries a phosphoethanolamine (PEA) substituent in position O-3 and in position O-6 or O-7; and (ii) the lipidated sub-unit B (TbpB) of the receptor of the human transferrine of an N. meningitidis strain or a lipid fragment of said TbpB.

The invention relates to the field of vaccines for combating infections caused by Neisseria meningitidis and especially proposes a vaccine composition comprising lipooligosaccharide (LOS) in association with the lipidized subunit B (TbpB) of the human transferrin receptor of a strain of N. meningitidis.

In the field of vaccines, one of the major challenges in the near future will especially be the marketing of a vaccine intended for preventing all the infections caused by N. meningitidis serogroup B. This bacterium is responsible for a certain number of pathologies, the dominant ones of which are meningitis and meningococcia, but also arthritis and pericarditis. Meningococcia may be complicated with purpura fulminans and fatal septic shock.

In general, meningitis is either of viral origin or of bacterial origin. In developed countries, the bacteria mainly responsible are: N. meningitidis and Streptococcus pneumoniae, which are involved, respectively, in about 40% and 50% of the cases of bacterial meningitis. In developing countries, Haemophilus influenzae also remains a significant source of meningitis.

In France, about 600 to 800 cases of N. meningitides-mediated meningitis are reported per year. In the United States, the number of cases rises to about 2500 to 3000 per year.

The species N. meningitidis is subdivided into serogroups according to the nature of the capsule polysaccharides. Although there are about a dozen serogroups, 90% of the cases of meningitis are attributable to the serogroups: A, B, C, Y and W135.

Efficient vaccines based on capsule polysaccharides exist for preventing the N. meningitidis-mediated meningitis of the serogroups A, C, Y and W135. These polysaccharides per se are not or are only sparingly immunogenic in children under 2 years old and do not induce any immune memory. However, these drawbacks may be overcome by combining these polysaccharides with a carrier protein.

However, the polysaccharide in capsule form of N. meningitidis serogroup B is not or only sparingly immunogenic in man, whether or not it is in conjugated form (Bruge et al, Vaccine (2004) 22: 1087). Moreover, this polysaccharide bears an epitope that might potentially undergo a cross reaction with human tissues. Thus, it appears highly desirable to search for a vaccine for combating meningitis induced by N. meningitidis especially of the serogroup B other than a vaccine based on capsule polysaccharide.

The liposaccharide (LPS) is a major constituent of the outer membrane of the wall of Gram-negative bacteria. LPS is toxic at high doses to mammals and, in view of this biological activity, has been called an endotoxin. It is responsible for septic shock, a fatal pathology which develops following acute infection with a Gram-negative bacterium.

Nevertheless, LPS is not only toxic, it is also immunogenic. In mammals, anti-LPS antibodies are generated during carrying and infection and can be protective. Thus, the use of LPS has already been envisioned in the prophylaxis of infections due to Gram-negative bacteria and associated diseases.

The structure of LPS is constituted of a lipid portion, called lipid A, covalently bonded to a polysaccharide portion.

Lipid A is responsible for the toxicity of LPS. It is highly hydrophobic and enables the LPS to be anchored in the outer membrane of the wall. Lipid A is composed of a disaccharide structure substituted with fatty acid chains. The number and the composition of the fatty acid chains vary from one species to the other.

The polysaccharide portion is constituted of carbohydrate chains which are responsible for the antigenicity. At least 3 major regions can be distinguished in this polysaccharide portion:

an inner core constituted of monosaccharides [one or more KDO (2-keto-3-deoxyoctulosonic acid) and one or more heptoses (Hep)] which are invariant within the same bacterial species;
(ii) an outer core bonded to heptose and constituted of various monosaccharides; and
(iii) an O-specific outer chain constituted of a series of repeating units—these repeating units themselves being composed of one or more different monosaccharides.

The structure of LPS varies from one species to another. This is why, for example, in a certain number of nonenteric Gram-negative bacteria such as Neisseriae, Bordetellae, Branhamellas, Haemophilus and Moraxellae, the O-specific chain does not exist. The LPS saccharide portion of these bacteria is constituted only of the oligosaccharide core. Consequently, the LPS from these bacteria is often called lipooligosaccharide (LOS).

The structure of the LPS varies not only from one species to another, but also within the same species.

Thus, not all the strains of N. meningitidis have an LOS of the same structure, although all the LOSs of meningococcus share the basic structure, which is represented schematically in the following structural formula:

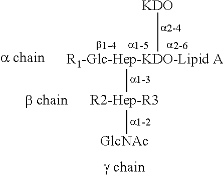

The outer core (or a chain) is variable as a function of the type of oligosaccharide (substituent R1), attached to the glucose residue borne by the heptose I.

Whereas lipid A is essentially invariable, the inner core, which itself also is formed in an invariant manner from two KDO (2-keto-3-deoxyoctulosonic acid) and two heptoses (HepI and HepII), bears various substituents (i) on heptose II (substituents R2 and R3); and (ii) on the γ chain, formed from an N-acetyl glucosamine (GlcNAc), which may or may not be O-acetylated. In the literature, the R2 residue is commonly referred to as the β chain, when R2 is a glucose residue.

The strains of N. meningitidis are classified into several immunotypes (IT L1 to L13), as a function of their reactivity with a series of antibodies which recognize the various epitopes on the LOS (Achtman et al, 1992, J. Infect. Dis. 165: 53-68). The majority of invasive strains with N. meningitidis serogroup B is of immunotype L3,7 as demonstrated by the reactivity of these strains with a monoclonal antibody called L3,7,9. This monoclonal antibody is capable of recognizing each of the immunotypes L3, L7 and L9 (Gu et al, J. Clin. Microbiol. (1992) 30: 2047; Moran et al, Infect. Immun. (1994) 62 (12): 5290; et Scholten et al, J. Med. Microbiol. (1994) 41: 236).

The classification of the LOS follows that of the strains. The differences between immunotypes originate from variations in the composition and in the conformation of the oligosaccharide chains. This shows in the table below, derived from Table 2 of Braun et al, Vaccine (2004) 22: 898, supplemented with data obtained subsequently and relating to immunotypes L9 (Schoudhury et al, Carbohydr. Res. (2008) 343: 2771) and L11 (Mistretta et al, (2008) Poster at the 16th International Pathogenic Neisseria Conference, Rotterdam):

TABLE I

| IT | R1 (α chain) | R2 | R3 |
|---|---|---|---|
| L1 | NeuNAcα2-6 Galα1-4 Galβ1-4 | PEA-3 | — |
| L2 | NeuNAcα2-3 Galβ1-4 GlcNAcβ1-3 | Glcα (1-3) | PEA-6 or |
| | Galβ1-4 | | PEA-7 |
| L3 | NeuNAcα2-3 Galβ1-4 GlcNAcβ1-3 | PEA-3 | — |
| | Galβ1-4 | | |
| L4 | NeuNAcα2-3 Galβ1-4 GlcNAcβ1-3 | — | PEA-6 |
| | Galβ1-4 | | |
| L5 | NeuNAcα2-3 Galβ1-4 GlcNAcβ1-3 | Glcα (1-3) | — |
| | Galβ1-4 Glcβ1-4 | | |
| L6 | GlcNAcβ1-3 Galβ1-4 | — | PEA-6 or |
| | | | PEA-7 |
| L7 | Galβ1-4 GlcNAcβ1-3 Galβ1-4 | PEA-3 | — |
| L8 | Galβ1-4 | PEA-3 | — |
| L9 | Galβ1-4 GlcNAcβ1-3 Galβ1-4 | — | PEA-6 |
| L10 | n.d. | n.d. | n.d. |
| L11 | Glcβ1-4 | PEA-3 | PEA-6. |
| L12 | n.d | n.d. | n.d. |
| L13 | n.d | n.d. | n.d. |

n.d.: not determined.

It may be noted, inter alia, that certain LOSs may be sialylated (presence of N-acetylneuraminic acid on the terminal galactose residue (Gal) of the a chain). Thus, immunotypes L3 and L7 differ only by the respective presence/absence of this sialylation. Moreover, most LOSs are substituted with an O-acetyl group on the glucosamine residue (α-GlcNAc) of the inner core (Wakarchuk et al. (1998) Eur. J. Biochem. 254: 626; Gamian et al. (1992) J. Biol. Chem. 267: 922; Kogan et al (1997) Carbohydr. Res. 298: 191; Di Fabio et al. (1990) Can. J. Chem. 68: 1029; Michon et al. (1990) J. Biol. Chem. 275: 9716; Choudhury et al. (above); and Mistretta et al. (above)).

The other variations in the structure of the LOS are due to different genetic factors, including:

(i) the presence/absence of certain genes involved in the LOS biosynthetic pathway and in possible mutual associations of the genes;
(ii) the phase variation to which certain genes are subjected;
(iii) homologous recombination [since certain genes have conserved regions (lgtB, lgtE and lgtH) and other genes are hybrid (lgtZ is the hybrid of the genes lgtA and lgtB), rearrangements may take place]; and
(iv) mutations.

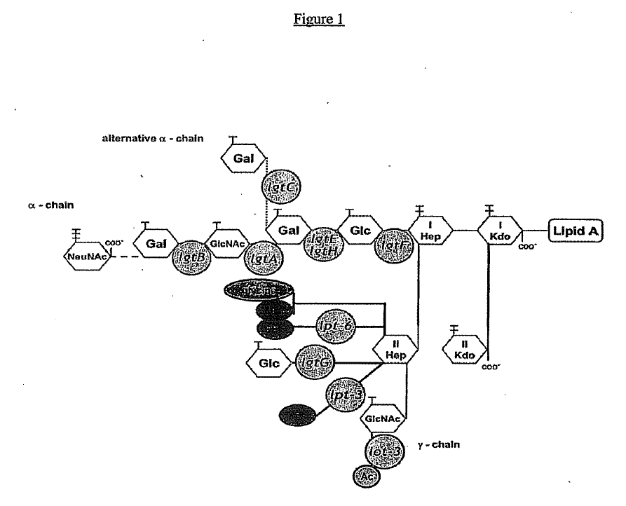

The genes involved in the LOS biosynthetic pathway (with the exception of two) are divided into three loci (lgt-1, lgt-2 and lgt-3). The description of these genes and their function is given later, illustrated schematically by FIG. 1, which shows the structure of the LOS of N. meningitidis, the various sites at which the variability is expressed and also the levels of intervention of the genes.

The off-locus genes are lpt3 and lot3. The gene lpt3 codes for a PEA transferase. This enzyme has the capacity to attach a phosphoethanolamine (PEA) residue in the 0-3 position of heptose II. The gene lot3 codes for an LOS O-acetyltransferase that has the capacity to O-acetylate the γ chain. It is subject to a phase variation.

The lgt-1 locus comprises 7 genes: lgtA, lgtB, lgtC, lgtD, lgtE, lgtH and lgtZ, each coding for a particular glycosyl transferase. Among these genes, lgtA and lgtC are subject to a phase variation. lgtE and lgtH have an allelic variation: the codon that determines the amino acid in position 153 codes either for a threonine residue (and in this case the resulting enzyme is a Gal-transferase) or for a methionine residue (and in this case the resulting enzyme is a Glc-transferase).

The lgt-1 locus is classed into 8 genetic types (Zhu et al, Microbiology (2002) 148: 1833).

The lgt-2 locus comprises 2 genes: lgtF and lgtK coding for glycosylases. The product of the lgtF gene intervenes in the construction of the a chain by enabling the binding of the glucose residue to heptose I, and therefore does not intervene in the nature of the immunotype; nor, for that matter, does the gene lgtK.

The lgt-3 locus comprises 2 genes: lgtG and lpt6. The gene lgtG codes for a Glc synthetase that has the capacity to attach a glucose residue in the 0-3 position of heptose II. The gene lpt6 codes for a PEA transferase that has the capacity to attach a phosphoethanolamine (PEA) substituent in the O-6 or O-7 position of heptose II. The gene lgtG is subject to a phase variation. When it is “On” and accompanied by a functional lpt3 gene, the attachment of the glucose residue always takes place at the expense of PEA (whose attachment is mediated by lpt3).

The lgt-3 locus is classed into 5 genetic types (Wright et al., J. Bact. (October 2004): 6970). The Galβ1-4 GlcNAcβ1-3 Galβ1-4Glcβ1-4 carbohydrate unit or lacto-N-neotetraose unit which is present in the a chain of certain N. meningitidis LOS immunotypes constitutes an epitope which can potentially crossreact with human erythrocytes. Thus, with a view to producing a vaccine for use in humans, it is advisable to choose an LOS which does not possess this unit. It may therefore be particularly advantageous to use an LOS originating from strains of immunotype L6 or L8.

Alternatively, it may also be envisaged to start, for example, with a strain of immunotype L2 or L3 in which a gene involved in the biosynthesis of the a chain has been inactivated by mutation, so as to obtain an incomplete structure LNnT. Such mutations are proposed in patent application WO 04/014417. The LOS of the mutated strains resulting therefrom have an a chain of L6 type and a phosphoethanolamine (PEA) substituent in position O-3 of the heptose II.

It has now been found that the LOS of a strain of immunotype L8 (LOS of immunotype L8), in combination with the lipidized subunit B (TbpB) of the human transferrin receptor of a strain of N. meningitidis can induce bactericidal activity that is improved relative to that induced by an LOS (in combination with the same TbpB) bearing an a chain of L6 type, and a phosphoethanolamine (PEA) substituent in position O-3 of the heptose II.

For this reason, the subject of the invention is a pharmaceutical vaccine composition for combating N. meningitidis infections, which comprises:
(i) an LOS of N. meningitidis formed especially from a lipid A, an inner core, an a chain of L8 type, in which the heptose II residue of the inner core (a) bears in position O-3 a phosphoethanolamine (PEA) substituent and does not bear a PEA substituent in positions O-6 and O-7; or (b) bears a phosphoethanol amine (PEA) substituent in position O-3 and in position O-6 or O-7; and
(ii) the lipidized subunit B (TpbB) of the human transferrin receptor of a strain of N. meningitidis or a lipidized fragment of this TpbB.

According to a particular aspect, a vaccine according to the invention comprises:
(i) an LOS of N. meningitidis formed especially from a lipid A, an inner core, an a chain of L8 type, in which the heptose II residue of the inner core bears in position O-3 a phosphoethanolamine (PEA) substituent and does not bear a PEA substituent in positions O-6 and O-7;
(ii) an LOS of N. meningitidis formed especially from a lipid A, an inner core, an a chain of L8 type, in which the heptose II residue of the inner core bears a phosphoethanolamine (PEA) substituent in position O-3 and in position O-6 or O-7; and
(iii) the TpbB of N. meningitidis or a lipidized fragment of the latter.

In the rest of the text, the term “or a lipidized fragment thereof” will no longer appear, for the sake of simplicity. Nevertheless, it remains implied each time that the term “lipidized TbpB” or “TbpB” appears.

A composition according to the invention may (i) prevent at least 60%, advantageously at least 70% and preferably at least 80%, of infections caused by N. meningitidis, especially of serogroup B or (ii) prevent infections that are at least 60%, advantageously at least 70% and preferably at least 80% caused by strains of N. meningitidis especially of serogroup B.

Advantageously, a composition according to the invention does not contain any OMV (outer membrane vesicle) of N. meningitidis.

### The LOS

For use in a composition according to the invention, the LOS formed especially from a lipid A, an inner core, an a chain of L8 type, in which the heptose II residue of the inner core bears a phosphoethanolamine (PEA) substituent in position O-3 and in position O-6 or O-7, may be that of an atypical strain of immunotype L8 such as the strain A1 (Zhu, Klutch & Tsai, FEMS Microbiology Letters (2001) 203: 173 and also Gu, Tsai & Karpas, J. Clin. Microbiol. (August 1992) 30 (8): 2047). According to one advantageous mode, the LOS is obtained from a strain of serogroup A.

It is also possible to manufacture a vaccine according to the invention, using an LOS formed especially from a lipid A, an inner core, an a chain of L8 type, in which the heptose II residue of the inner core bears in position O-3 a phosphoethanolamine (PEA) substituent and does not bear a PEA substituent in positions O-6 and O-7. Typically, such an LOS is obtained from a strain of immunotype L8, preferably of serogroup A. It is also proposed to obtain such an LOS from a strain of N. meningitidis of immunotype L6 modified such that it expresses a gene lpt3 and such that it no longer expresses the genes lpt6 and lgtA. The starting strain which can be used for modification purposes may be the strain C708 filed on 11 Mar. 2008 at the Collection Nationale de Culture de Microorganisme, 25 rue du Dr Roux 75015 Paris, according to the terms of the treaty of Budapest. This strain bears the order number CNCM I-3942. This strain bears, inter alia, an active lgtA gene (gene switched “ON”); an lgtB gene—(non-functional gene); an lgtG gene (switched “Off”); a truncated lpt3 gene; an active lpt6 gene; an active lot3 gene; and an active msbB gene.

The strain C708 comprises a truncated lpt3 gene. To modify it such that the LOS bears a PEA substituent in position O3, the functionality of the lpt3 gene may be restored, especially by homologous recombination using a complete (full-length) lpt3 gene. When this strain is used in the process according to the invention, it is also appropriate to deactivate the lptA and lpt6 genes so as to obtain a strain that no longer expresses these genes. The deactivation of these genes may especially be achieved by total or partial deletion of the lptA and lpt6 genes or alternatively by insertion of a non-pertinent sequence into the gene, for example an antibiotic-resistant gene.

The N. meningitidis serogroup against which it is imperative to propose a vaccine in priority is the serogroup B (vaccines against the other prevalent serogroups A, C, Y and W135 are already available). Now, purification of the LOS from a strain of serogroup B may lead to a product containing residual amounts of capsule polysaccharide which are in the vaccine. To overcome these difficulties, it has now been found that an ad hoc LOS derived from a strain of serogroup A can satisfy the needs in terms of vaccination against the serogroup B.

Advantageously, an LOS for use in a composition according to the invention bears a γ chain that is O-acetylated, at least partially.

For the purposes of the present invention, the LOS can be obtained by conventional means: in particular it can be extracted from a bacterial culture; then purified according to standard methods. Many production processes are described in the literature. By way of example, mention is made inter alia of Westphal & Jann, (1965) Meth. Carbohydr. Chem. 5:83; Gu & Tsai, 1993, Infect. Immun. 61 (5): 1873; Wu et al., 1987, Anal. Biochem. 160: 281 and U.S. Pat. No. 6,531,131. An LOS preparation can be quantified according to well-known procedures. Assaying the KDO by high performance anion exchange chromatography HPAEC-PAD is a particularly suitable method.

### Detoxification of the LOS

For incorporation into a vaccine, the LOS needs to be detoxified. The toxicity of the LOS is due to its lipid A. However, it is not imperative to remove the lipid A in its entirety; nor to modify it, for example, by mutation (e.g. msbB minus mutation). In fact, since the toxicity is more particularly linked to a supramolecular conformation conferred by all the fatty acid chains borne by the disaccharide nucleus of the lipid, according to one advantageous embodiment, it is sufficient to act on these chains.

The detoxification can be obtained according to various approaches: chemical, enzymatic or genetic or alternatively by complexation with a peptide analog of polymyxin B or alternatively by incorporation/formulation into liposomes.

The level of detoxification of the LOS can be assessed inter alia according to one of the two following standard tests:
the pyrogenic test in rabbits. This test, the calculations and the reading thereof have been implemented according to the principles set out in the European Pharmacopea (Edition 6.0, paragraph 2.6.8.).
the LAL test (Limulus Amebocyte Lysate) implemented according to the principles set out in the European Pharmacopea (Edition 6.0, paragraph 2.6.14.).

### Detoxification Via the Chemical Route

The chemical approach consists in treating the LOS with a chemical agent. According to one particular embodiment, the LOS is subjected to mild acid hydrolysis with acetic acid which removes the lipid A and also the branched KDO(s) when it (they) is (are) present in the LOS structure. Such a treatment is, for example, described in Gu & Tsai Infect. Immun. (1993) 61: 1873. According to an alternative and preferred embodiment, the LOS is subjected to a de-O-acylation, preferably a primary de-O-acylation, i.a. by treatment with hydrazine, which hydrolyzes the esterified primary fatty acid chains of the lipid A. Such a treatment is, for example, described in U.S. Pat. No. 6,531,131, Gupta et al, Infect. Immun. (1992) 60 (8): 3201 and Gu et al, Infect. Immun. (1996) 64 (10): 4047.

### Detoxification Via the Enzymatic Route

The enzymatic approach consists in placing the LOS in the presence of lipases capable of digesting the esterified fatty acid chains of the lipid A. Such lipases are produced by the amoeba Dictyostelium discoideum. According to one particularly advantageous embodiment, the amoeba and a Gram-negative bacterium that can be phagocytosed by the amoeba, such as N. meningitidis, are cultured together (coculture). The supernatant is then recovered and the LOS is extracted from the supernatant which is then free of fatty acid chains. It may also be an acyloxyacyl hydrolase produced by certain human cells (patent WO 87/07297 Munford R.) or by Salmonella typhimurium (Trent et al 2001 J. Biol. Chem. 276: 9083-9092; Reynolds et al. 2006 J. Biol. Chem. 281: 21974-21987) (enzyme encoded by the PagL or LpxR genes in the latter case).

### Detoxification Via the Genetic Route

The genetic approach consists in using an LOS produced by a bacterial strain of which the genotype is such that the entity of the LOS normally responsible for its toxicity (lipid A, and more particularly the lipid tails of lipid A) has a greatly reduced or even nonexistent degree of toxicity. Such a bacterial strain can be conveniently obtained by mutation. Starting from a wild-type strain (i.e. a strain producing a toxic LOS), this then involves inactivating, by mutation, certain genes involved in the biosynthesis of the fatty acid chains, or in the attachment thereof to the disaccharide nucleus of lipid A. Thus, it is possible to envision inactivating the lpxL1 or lpxL2 genes (also called htrB1/htrB2) of N. meningitidis or equivalents thereof in other species (for example, the equivalents of the meningococcal lpxL1 and lpxL2 genes are respectively called msbB or lpxM and htrB or lpxL in E. coli.). A mutation that inactivates one of these genes results in an LOS devoid of one or of two secondary acyl chains. lpxL1 or L2 mutants of N. meningitidis or of Haemophilus influenzae are in particular described in patent applications WO 00/26384, US 2004/0171133 and WO 97/019688. In N. meningitidis, the endogenous lpxA gene can also be replaced with the homologous gene originating from E. coli or Pseudomonas aeruginosa. The fatty acid chains thereof are modified, resulting in a less toxic lipid A (Steeghs et al, Cell. Microbiol. (2002) 4 (9): 599). The genetic approach is favored when the LOS is purified in the form of OMVs.

Los Detoxified by Complexation with a Peptide Analog of Polymyxin B

A fourth approach consists in complexing the LOS with a peptide analog of polymyxin B, as is, for example, described in patent application WO 06/108586. The LOS that is complexed and consequently detoxified is called endotoxoid.

The polymyxin B analog included in the composition of an endotoxoid that is useful for the purposes of the present invention may be any peptide that is capable of detoxifying the LOS by simple complexation. Such peptides are especially described in patent or patent applications U.S. Pat. No. 6,951,652, EP 976 402 and WO 06/108 586.

Thus, an advantageous peptide may be the peptide of formula (I) NH₂-A-Cys1-B-Cys2-C—COOH, in which:
A is a peptide of 2 to 5 and preferably 3 or 4 amino acid residues, in which at least 2 amino acid residues are independently chosen from Lys, Hyl (hydroxylysine), Arg and His;
B is a peptide of 3 to 7 and preferably 4 or 5 amino acid residues, which comprises at least two and preferably three amino acid residues chosen from Val, Leu, Ile, Phe, Tyr and Trp; and
C is optional (this position may or may not be empty) and is an amino acid residue or a peptide formed from 2 to 3 amino acid residues;
   on condition that the cationic amino acid/hydrophobic amino acid ratio in the peptide of formula I is from 0.4 to 2, advantageously from 0.5 to 1.2 or 1.5, preferably from 0.6 to 1; better still from 0.6 to 0.8; for example 0.75.

Preferably, in the peptide of formula (I), position C is an empty position.

Particular examples of the peptide of formula (I) are the following peptides:
NH₂-Lys-Thr-Lys-Cys1-Lys-Phe-Leu-Lys-Lys-Cys2-COOH (peptide SAEP2);
NH₂-Lys-Thr-Lys-Cys1-Lys-Phe-Leu-Leu-Leu-Cys2-COOH (peptide SAEP2-L2);
NH₂-Lys-Arg-His-Hyl-Cys1-Lys-Arg-Ile-Val-Leu-Cys2-COOH;
NH₂-Lys-Arg-His-Cys1-Val-Leu-Ile-Trp-Tyr-Phe-Cys2-COOH;
NH₂-Lys-Thr-Lys-Cys1-Lys-Phe-Leu-Leu-Leu-Cys2-COOH; and
NH₂-Hyl-Arg-His-Lys-Cys1-Phe-Tyr-Trp-Val-Ile-Leu-Cys2-COOH.

The peptides of formula (I) may be in monomer form or, preferably, in parallel or antiparallel dimer form.

In general, use may also be made of a dimeric peptide of formula (II)

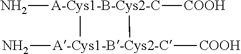

in which the two Cys1 residues are linked together via a disulfide bridge and the two Cys2 residues are linked together via a disulfide bridge;
or of formula (III)

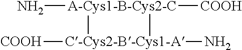

in which the Cys1 residues are linked to the Cys2 residues via peptide inter-chain disulfide bridges;

in which formulae (II) and (III):
A and A′ are, independently, a peptide of 2 to 5 and preferably 3 or 4 amino acid residues, in which at least 2 amino acid residues are independently chosen from Lys, Hyl (hydroxylysine), Arg and His;
B and B′ are, independently, a peptide of 3 to 7 and preferably 4 or 5 amino acid residues, which comprise at least two and preferably three amino acid residues independently chosen from Val, Leu, Ile, Phe, Tyr and Trp; and
C and C′ are optional (these positions may or may not be empty) and are, independently, an amino acid residue or a peptide of 2 to 3 amino acid residues; on condition that the cationic amino acid/hydrophobic amino acid ratio in the dimer of formula (II) or (III) is from 0.4 to 2, advantageously from 0.5 to 1.2 or 1.5, preferably from 0.6 to 1 and better still from 0.6 to 0.8; for example. 0.75.

Advantageously, A and A′ are, independently, a peptide of 2 to 5 and preferably 3 or 4 amino acid residues, in which at least one and preferably 2 amino acid residues are independently chosen from Lys, Hyl, Arg and His; and, where appropriate, those that are not chosen from Lys, Hyl, Arg and His (“the remaining amino acid residues”) being chosen from the group of uncharged, polar or nonpolar amino acid residues; preferably Thr, Ser and Gly; most particularly preferably Thr.

When A and A′ comprise 3 amino acid residues, each of them may be a cationic residue; or alternatively, two of the three residues are cationic amino acids, whereas the remaining residue is chosen from the group of uncharged, polar or nonpolar amino acid residues; preferably Thr, Ser and GIy; most particularly preferably Thr.

When A and A′ comprise 4 amino acid residues, it is preferable for two or three of the four residues to be chosen from the groups of cationic amino acid residues as defined above, whereas the remaining residue(s) is (are) chosen from the group of uncharged, polar or nonpolar amino acid residues as defined above.

When A and A′ comprise 5 amino acid residues, it is preferred for three or four of the five residues to be chosen from the groups of cationic amino acid residues as defined above, whereas the remaining residue(s) is (are) chosen from the group of uncharged, polar or nonpolar amino acid residues as defined above.

Advantageously, B and B′ are, independently, a peptide of 3 to 7 and preferably 4 or 5 amino acid residues, which comprises at least two and preferably three amino acid residues independently chosen from Val, Leu, Ile, Phe, Tyr and Trp; preferably Leu, Ile and Phe; and, where appropriate, those that are not chosen from Val, Leu, Ile, Phe, Tyr and Trp (“the remaining amino acid residues”) being chosen independently from the group formed by Lys, Hyl, Arg and His. As may readily be understood, B and B′ may comprise up to 7 amino acid residues independently chosen from Val, Leu, Ile, Phe, Tyr and Trp.

Advantageously, B and B′ comprise the sequence -X1-X2-X3-, in which X1 and X2; X2 and X3; or X1, X2 and X3 are independently chosen from Val, Leu, Ile, Phe, Tyr and Trp; preferably from Leu, Ile and Phe. In one preferred embodiment, the sequence -X1-X2-X3- comprises the Phe-Leu unit.

The particular embodiments of B and B′ include:

(i) the sequence -X1-X2-X3- in which:
X1 is Lys, Hyl, His or Arg, preferably Lys or Arg; preferably Lys;
X2 is Phe, Leu, Ile, Tyr, Trp or Val; preferably Phe or Leu; more particularly preferably Phe; and
X3 is Phe, Leu, Ile, Tyr, Trp or Val; preferably Phe or Leu; more particularly preferably Leu; and
(ii) where appropriate, the amino acid residues are each independently chosen from the group formed by Val, Leu, Ile, Phe, Tyr, Trp, Lys, Hyl, Arg and His; preferably Val, Leu, Ile, Phe, Tyr and Trp; more particularly preferably Leu, Ile and Phe.

When B and B′ comprise more than 4 nonpolar amino acid residues, A and A′ preferably comprise at least 3 positively charged amino acid residues.

In C and C′, the amino acid residues may be any amino acid residue, on condition that the cationic amino acid residues/hydrophobic amino acid residues ratio remains in the indicated range. Advantageously, they are independently chosen from uncharged, polar or nonpolar amino acid residues, the latter being preferred. However, preferably, the positions C and C′ are empty positions.

Consequently, a preferred class of the dimers is of formula (IV)

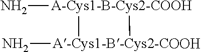

in which the two Cys1 residues are linked together via a disulfide bridge and the two Cys2 residues are linked together via a disulfide bridge;

or of formula (V)

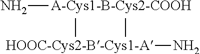

in which the Cys1 residues are linked to the Cys2 residues via peptide inter-chain disulfide bridges;

in which formulae (IV) and (V), in which A, A′, B and B′ are as described above; on condition that the cationic amino acid/hydrophobic amino acid ratio in the dimer of formula (IV) or (V), is from 0.4 to 2, advantageously from 0.5 to 1.2 or 1.5, preferably from 0.6 to 1 and better still from 0.6 to 0.8; for example 0.75.

In formulae (II) to (V), A and A′ are preferably identical. This is likewise the case as regards B and B′, on the one hand, and C and C′, on the other hand. A dimer of formula (II) to (V), in which A and A′; B and B′; and C and C′ are identical in pairs, is designated as a homologous dimer.

For these dimers, mention may be made, for example, of the parallel and antiparallel dimers formed from the peptide SAEP2-L2:

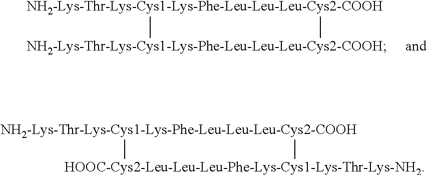

The endotoxoid that is useful for the purposes of the present invention may advantageously be characterized by an LOS/peptide mole ratio from 1/1.5 to 1/0.5, preferably from 1/1.2 to 1/0.8, and most particularly preferably from 1/1.1 to 1/0.9, e.g. 1/1.

### Los in Detoxified Liposomes

When the LOS is formulated in liposomes, it does not appear to be necessarily required to detoxify it beforehand. This is because LOS in liposomes—i.e. associated with the lipid bilayer forming the liposomes—may experience a very substantial decrease in toxicity. The size of this decrease, which can be as much as a substantial loss, depends partly on the nature of the components forming the liposome. Thus, when positively charged components (components of cationic nature) are used, the loss of toxicity may be greater than with uncharged (neutral) or anionic components.

The term “liposomes” is intended to mean a synthetic entity, preferably a synthetic vesicle, formed of at least one lipid bilayer membrane (or matrix) enclosing an aqueous compartment. For the purposes of the present invention, the liposomes may be unilamellar (a single bilayer membrane) or multilamellar (several membranes layered like an onion). The lipids constituting the bilayer membrane comprise a nonpolar region which, typically, is made of chain(s) of fatty acids or of cholesterol, and a polar region, typically made of a phosphate group and/or of tertiary or quaternary ammonium salts. Depending on its composition, the polar region may, in particular at physiological pH (pH≈7) carry either a negative (anionic lipid) or positive (cationic lipid) net (overall) surface charge, or not carry a net charge (neutral lipid).

For the purposes of detoxifying the LOS, the liposomes may be liposomes of any type; in particular, they may be constituted of any lipid known to be of use in the production of liposomes. The lipid(s) that go(es) to make up the composition of the liposomes may be neutral, anionic or cationic lipid(s); the latter being preferred. These lipids may be of natural origin (plant or egg extraction products, for example) or synthetic origin; the latter being preferred. The liposomes may also be constituted of a mixture of these lipids; for example, of a cationic or anionic lipid and of a neutral lipid, as a mixture. In the latter two cases, the neutral lipid is often referred to as colipid. According to one advantageous mixture embodiment, the charged (cationic or anionic) lipid: neutral lipid mole ratio is between 10:1 and 1:10, advantageously between 4:1 and 1:4, preferably between 3:1 and 1:3, limits included.

With regard to the neutral lipids, mention is made, by way of example, of: (i) cholesterol; (ii) phosphatidylcholines such as, for example, 1,2-diacyl-sn-glycero-3-phosphocholines, e.g. 1,2-dioleoyl-sn-glycero-3-phosphocholine (DOPC), and also 1-acyl-2-acyl-sn-glycero-3-phosphocholines of which the acyl chains are different than one another (mixed acyl chains); and (iii) phosphatidylethanolamines such as, for example, 1,2-diacyl-sn-glycero-3-phosphoethanolamines, e.g. 1,2-dioleoyl-sn-glycero-3-phosphoethanolamine (DOPE), and also 1-acyl-2-acyl-sn-glycero-3-phospho-ethanolamines bearing mixed acyl chains.

With regard to the anionic lipids, mention is made, by way of example, of: (i) cholesteryl hemisuccinate (CHEMS); (ii) phosphatidylserines such as 1,2-diacyl-sn-glycero-3-[phospho-L-serine]s, e.g. 1,2-dioleoyl-sn-glycero-3-[phospho-L-serine] (DOPS), and 1-acyl-2-acyl-sn-glycero-3-[phospho-L-serine]s bearing mixed acyl chains; (iii) phosphatidylglycerols such as 1,2-di acyl-sn-glycero-3-[phospho-rac-(1-glycerol)]s, e.g. 1,2-dioleoyl-sn-glycero-3-[phospho-rac-(1-glycerol)] (DOPG), and 1-acyl-2-acyl-sn-glycero-3-[phospho-rac-(1-glycerol)]s bearing mixed acyl chains; (iv) phosphatidic acids such as 1,2-diacyl-sn-glycero-3-phosphates, e.g. 1,2-dioleoyl-sn-glycero-3-phosphate (DOPA), and 1-acyl-2-acyl-sn-glycero-3-phosphates bearing mixed acyl chains; and (v) phosphatidylinositols such as 1,2-diacyl-sn-glycero-3-(phosphoinositol)s, e.g. 1,2-dioleoyl-sn-glycero-3-(phosphoinositol) (DOPI), and 1-acyl-2-acyl-sn-glycero-3-(phosphoinositol)s bearing mixed acyl chains.

With regard to the cationic lipids, mention is made, by way of example, of:

(i) lipophilic amines or alkylamines such as, for example, dimethyldioctadecylammonium (DDA), trimethyldioctadecylammonium (DTA) or structural homologs of DDA and of DTA [these alkylamines are advantageously used in the form of a salt; mention is made, for example, of dimethyldioctadecylammonium bromide (DDAB)];
(ii) octadecenoyloxy(ethyl-2-heptadecenyl-3-hydroxyethyl)imidazolinium (DOTIM) and structural homologs thereof;
(iii) lipospermines such as N-palmitoyl-D-erythrosphingosyl-1-O-carbamoylspermine (CCS) and dioctadecylamidoglycylspermine (DOGS, transfectam);
(iv) lipids incorporating an ethylphosphocholine structure, such as cationic derivatives of phospholipids, in particular phosphoric ester derivatives of phosphatidylcholine, for example those described in patent application WO 05/049080 and including, in particular:
1,2-dimyristoyl-sn-glycero-3-ethylphosphocholine,
1,2-dipalmitoyl-sn-glycero-3-ethylphosphocholine,
1,2-palmitoyloleoyl-sn-glycero-3-ethylphosphocholine,
1,2-distearoyl-sn-glycero-3-ethylphosphocholine (DSPC),
1,2-dioleyl-sn-glycero-3-ethylphosphocholine (DOEPC or EDOPC or ethyl-DOPC or ethyl PC),
and also structural homologs thereof;
   (v) lipids incorporating a trimethylammonium structure, such as N-(1-[2,3-dioleyloxy]propyl)-N,N,N-trimethylammonium (DOTMA) and structural homologs thereof and those incorporating a trimethylammonium propane structure, such as 1,2-dioleyl-3-trimethylammonium propane (DOTAP) and structural homologs thereof; and also lipids incorporating a dimethylammonium structure, such as 1,2-dioleyl-3-dimethylammonium propane (DODAP) and structural homologs thereof; and
   (vi) cationic derivatives of cholesterol, such as 3β-[N-(N′,N′-dimethylaminoethane)-carbamoyl] cholesterol (DC-Chol) or other cationic derivatives of cholesterol, such as those described in U.S. Pat. No. 5,283,185, and in particular cholesteryl-3,3-carboxamidoethylenetrimethylammonium iodide, cholesteryl-3β-carboxyamidoethylene-amine, cholesteryl-3β-oxysuccinamidoethylenetrimethylammonium iodide and 3β-[N-(polyethyleneimine)carbamoyl]cholesterol.

The term “structural homologs” signifies lipids which have the characteristic structure of the reference lipid while at the same time differing therefrom by virtue of secondary modifications, especially in the nonpolar region, in particular of the number of carbon atoms and of double bonds in the fatty acid chains.

These fatty acids, which are also found in the neutral and anionic phospholipids, are, for example, dodecanoic or lauric acid (C12:0), tetradecanoic or myristic acid (C14:0), hexadecanoic or palmitic acid (C16:0), cis-9-hexadecanoic or palmitoleic acid (C16:1), octadecanoic or stearic acid (C18:0), cis-9-octadecanoic or oleic acid (C18:1), cis,cis-9,12-octadecadienoic or linoleic acid (C18:2), cis-cis-6,9-octadecadienoic acid (C18:2), all-cis-9,12,15-octadecatrienoic or α-linolenic acid (C18:3), all-cis-6,9,12-octadecatrienoic or γ-linolenic acid (C18:3), eicosanoic or arachidic acid (C20:0), cis-9-eicosenoic or gadoleic acid (C20:1), all-cis-8,11,14-eicosatrienoic acid (C20:3), all-cis-5,8,11,14-eicosatetraenoic or arachidonic acid (C20:4), all-cis-5,8,11,14,17-eicosapentaneoic acid (C20:5), docosanoic or behenic acid (C22:0), all-cis-7,10,13,16,19-docosapentaenoic acid (C22:5), all-cis-4,7,10,13,16,19-docosahexaenoic acid (C22:6) and tetracosanoic or lignoceric acid (C24:0).

According to one particular embodiment, a mixture of cationic lipid and neutral lipid is used. By way of example, mention is made of
a mixture of DC-chol and DOPE, in particular in a DC-chol:DOPE mole ratio ranging from 10:1 to 1:10, advantageously from 4:1 to 1:4, preferably from approximately 3:1 to 1:3;
a mixture of ethyl-DOPC and cholesterol, in particular in an ethyl-DOPC:cholesterol mole ratio ranging from 10:1 to 1:10, advantageously from 4:1 to 1:4, preferably from approximately 3:1 to 1:3; and
a mixture of ethyl-DOPC and DOPE, in particular in an ethyl-DOPC:DOPE mole ratio ranging from 10:1 to 1:10, advantageously from 4:1 to 1:4, preferably from approximately 3:1 to 1:3.

According to one advantageous method of preparation, in an initial step, a dry lipid film is prepared with all the compounds that go to make up the composition of the liposomes. The lipid film is then reconstituted in an aqueous medium, in the presence of LOS, for example in a lipid:LOS mole ratio of 100 to 500, advantageously of 100 to 400, preferably of 200 to 300, most particularly preferably of approximately 250. In general, it is considered that this same mole ratio should not substantially vary at the end of the method of preparing the LOS liposomes.

In general, the reconstitution step in an aqueous medium results in the spontaneous formation of multilamellar vesicles, the size of which is subsequently homogenized by gradually decreasing the number of lamellae by extrusion, for example using an extruder, by passing the lipid suspension, under a nitrogen pressure, through polycarbonate membranes having decreasing pore diameters (0.8, 0.4, 0.2 μm). The extrusion process can also be replaced with another process using a detergent (surfactant) which disperses lipids. This detergent is subsequently removed by dialysis or by adsorption onto porous polystyrene microbeads with a particular affinity for detergent (BioBeads). When the surfactant is removed from the lipid dispersion, the lipids reorganize in a double layer.

At the end of the incorporation of the LOS into liposomes, a mixture constituted of ad hoc liposomes and of LOS in free form may commonly be obtained. Advantageously, the liposomes are then purified in order to be rid of the non-detoxified LOS in free form.

### Conjugation of LOS

In a vaccine according to the invention, the LOS is advantageously in the form of a LOS/polypeptide carrier conjugate, in particular when it is not in the form of OMVs or liposomes.

The carrier polypeptide can be any carrier polypeptide, oligopeptide or protein in use in the conjugated vaccines field; and in particular pertussis, diphtheria or tetanus toxoid, the diphtheria toxin mutant named CRM197, a bacterial OMP, a protein bacterial complex [for example, N. meningitidis OMPC (outer-membrane protein C)], Pseudomonas exotoxin A, Haemophilus influenzae lipoprotein D, Streptococcus pneumoniae pneumolysin, Bordetella pertussis filamentous hemagglutinin and the lipidized subunit B of the N. meningitidis human transferrin receptor.

Many methods of conjugation exist in the technical field. Some are listed, for example, in patent applications EP 941 738 and WO 98/31393.

In general, the reactive groups of the LOS involved in the conjugation are those of the inner core or of lipid A. It may involve, inter alia, the acid function of the KDO, or else an aldehyde generated subsequent to an appropriate treatment on the disaccharide of lipid A. For example, a phosphatase treatment generates an aldehyde on the structure of the second glucosamine of lipid A from N. meningitidis (Brade H. (2002) J. Endotoxin Res. 8 (4): 295 Mieszala et al, (2003) Carbohydrate Res. 338: 167 and Cox et al, (2005) Vaccine 23 (5): 5054).

Advantageously, the method of conjugation makes use (i) of a bifunctional linking agent (linker) or (ii) of a spacer and of a linker.

For example, in the first case, the LOS is activated with a bifunctional coupling agent (linker) of formula R1-A-R2, such that the R2 radical reacts with a reactive group of the KDO or of the lipid A in order to obtain an activated LOS; the activated LOS is then conjugated with the polypeptide such that the R1 substituent reacts with a functional group borne by the polypeptide, in order to obtain a conjugate.

For example, in the second case, the LOS is derivatized with a spacer of formula R3-B-R4 such that the R3 radical reacts with a reactive group of the KDO or of the lipid A in order to obtain a derivatized LOS; the derivatized LOS is then activated with a bifunctional coupling agent (linker) of formula R1-A-R2 such that the R2 radical reacts with the R4 radical in order to obtain a derivatized and activated LOS; finally, the derivatized and activated LOS is conjugated with the polypeptide such that the R1 radical reacts with a functional group borne by the polypeptide in order to obtain a conjugate.

In the second case, the process can also be carried out in the following way: the polypeptide is derivatized with a spacer of formula R3-B-R4 such that the R4 radical reacts with a functional group borne by the polypeptide; the LOS is activated with a bifunctional linker of formula R1-A-R2 such that the R2 radical reacts with a reactive group of the KDO or of the lipid A, in order to obtain an activated LOS; and then the activated LOS is conjugated with the derivatized polypeptide such that the R1 radical of the activated LOS reacts with the R3 radical of the derivatized polypeptide, in order to obtain a conjugate.

In the formula of the spacer, B may be a carbon chain, preferably carbonyl, alkyl or alkylene, for example C1 to C12. R3 and R4 may independently be a thiol or amine group or a residue bearing same, for example a hydrazide group, i.e. NH₂—NH—CO—. Compounds that may be used as a spacer have, for example, the formula NH₂—B—NH₂, or preferably NH₂—B—SH and NH₂—B—S—S—B′—NH₂. By way of particular example, mention is made of: cysteamine, cysteine, diamines, e.g. diaminohexane, adipic acid dihydrazide (ADH), urea and cystamine.

In the formula of the linker, A may be an aromatic or preferably aliphatic chain which is substituted or unsubstituted and which advantageously contains from 1 to 12 carbon atoms, preferably 3 to 8 carbon atoms. For example, A may be a C2 to C8 alkylene, a phenylene, a C7 to C12 aralkylene, a C2 to C8 alkyl, a phenyl, a C7 to C12 aralkyl, a C6 alkanoyloxy or a benzylcarbonyloxy, which may be substituted or unsubstituted.

The R2 radical is the functional group of the linker which creates the link with the LOS or with the derivatized LOS. Thus, R2 is a functional group which can react with a carboxyl, hydroxyl, aldehyde or amine group. If the linker must react with a hydroxyl, carboxyl or aldehyde group, R2 is preferably an amine group or a residue carrying an amine group, for example a hydrazide group, i.e. NH₂—NH—CO—. If the linker must react with an amine group, R2 is preferably a carboxyl, succinimidyl (e.g. N-hydroxy-succinimidyl) or sulfosuccinimidyl (e.g. N-hydroxysulfosuccinimidyl) group. Thus, compounds that can be used as a linker may be chosen from adipic acid dihydrazide (ADH); sulfosuccinimidyl 6-(3-[2-pyridyldithio]propionamido)hexanoate (Sulfo-LC-SPDP); succinimidyl 6-(3-[2-pyridyldithio]propionamido)hexanoate (LC-SPDP); N-succinimidyl-5-acetyl thioacetate (SATA); N-succinimidyl 3-(2-pyridyldithio)propionate (SPDP), succinimidyl acetylthiopropionate (SATP); succinimidyl-4-(N-maleimidomethyl)cyclohexane-1-carboxylate (SMCC); maleimidobenzoyl-N-hydroxysuccinimide ester (MBS); N-succinimidyl (4-iodoacetyl)aminobenzoate (SIAB); succinimidyl 4-(p-maleimidophenyl)butyrate (SMPB); bromoacetic acid-N-hydroxysuccinimide (BANS) ester; dithiobis-(succinimidylpropionate) (DTSSP); H-(γ-maleimidobutyryloxy)succinimide ester (GMBS); succinimidyl-4-(N-maleimidomethyl)cyclohexane-1-carboxylate; N-succinimidyl-4-(4-maleimidophenyl)butyrate; N-M-maleimidocaproic acid]hydrazide (BMCH); N-succinimidyl-4-maleimidobutyrate; and N-succinimidyl-3-maleiimidobenzoate.

By way of example, it is proposed to use the acid function of the KDO in order to derivatize the LOS with ADH in the presence of a carbodiimide [e.g. 3-ethyl-(3-dimethylaminopropyl)carbodiimide hydrochloride (EDAC)]. The amine function thus introduced is then reacted with the carboxyl functions of the polypeptide, in the presence of EDAC, after having protected the amine functions of the latter (Wu et al (2005) Vaccine 23: 5177) or having converted them to acid functions (succinylation of the protein; Pavliakova et al, Infect. Immun. (1999) 67 (10): 5526).

Alternatively, it is proposed to use the acid function of the KDO in order to derivatize the LOS with cysteamine or cysteine in the presence of EDAC. The thiol function thus introduced is then reacted with the maleimide function of a homobifunctional linker, such as bismaleimidohexane; or a heterobifunctional linker, such as GMBS. In the first case, the maleimide function thus introduced is then reacted with the thiol functions of the polypeptide. In the second case, the succinimidyl function of the derivatized and activated LOS is reacted with the amine functions of the polypeptide.

Depending on the method of conjugation selected, the LOS and the polypeptide can be conjugated to one another in an LOS:polypeptide mole ratio of from 10⁻¹ to 10², advantageously from 1 to 10², preferably from 1 to 50; most particularly preferably of approximately 20.

The TbpB of N. meningitidis

The TbpB of N. meningitidis as naturally produced by N. meningitidis is a lipoprotein. However, it may be advantageously produced in a recombinant manner in an expression system that makes it possible especially to ensure the lipidation of the polypeptide within the very organism responsible for the expression. According to one preferred mode, the lipidized TbpB is a recombinant TbpB—i.e. produced in a recombinant manner, e.g. in a heterologous expression system.

An expression system typically uses an expression cassette and a prokaryotic or eukaryotic host cell (yeast). The expression cassette codes for a TbpB precursor (also known as pro-'TbpB). This precursor is formed from a signal sequence characteristic of a lipoprotein and of the sequence of the mature protein containing a cysteine residue in the N-terminal position. The three amino acids in the C-terminal position of the signal sequence and the cysteine residue in the N-terminal position of the mature sequence constitutes the site of cleavage (also known as the lipobox): this lipobox typically has the sequence: Leu-Ser/Ala-Ala/Gly-Cys. A typical signal sequence is that of the lipoprotein Lpp of E. coli: Met-Lys-Ala-Thr-Lys-Leu-Val-Leu-Gly-Ala-Val-Ile-Leu-Gly-Ser-Thr-Leu-Leu-Ala-Gly. Thus, in the expression cassette, the polynucleotide sequence coding for the amino acid sequence of TbpB is 5′-fused to an appropriate signal sequence.

The TbpB of N. meningitidis is, like any protein, defined by a sequence of amino acids. Within the species, this amino acid sequence may have a certain degree of variability without this harming the biological function of the lipoprotein. This is then referred to as an “allelic variant”. A TbpB of N. meningitidis has a multiplicity of corresponding sequences having between themselves a certain degree of identity, each of the sequences originating from a particular strain, one being the allelic variant of the other.

Thus, it will be easily understood that the present invention is not limited to the use of a particular TbpB, defined by a particular amino acid sequence. Any reference to an amino acid sequence is given as a nonlimiting illustration.

The present invention is not limited either to a wild-type lipidized TbpB. Specifically, it may be a case not only of a wild-type form, but also of a form mutated by addition, substitution or deletion of one or more amino acids.

For use in the present invention, a lipidized fragment of TbpB of N. meningitidis is advantageously the lipidized N-terminal fragment of TbpB. The “polypeptide” part of the fragment may advantageously comprise one or more T-helper epitopes—i.e. epitopes that are capable of being recognized by T-helper cells—and of activating them. Advantageously, they are T-helper epitopes that are characteristic of the organism for which the LOS-based vaccine is intended (a mammal, especially a human)—i.e. epitopes that are capable of being recognized by the T-helper cells of the receiving organism and of activating them.

The open reading frame (ORF) coding for the TbpB (tbpB) of several strains of N. meningitidis has already been identified by its sequence, and the amino acid sequence of the corresponding protein has been deduced. Thus, the tbpB and TbpB sequences of the strains of N. meningitidis of serogroup B, M982 and B16B6 are disclosed in patent application EP 586 266. Those of the strains of meningococcus MC58 (serogroup B), Z2491 (serogroup A) and FAM18 (serogroup C) are disclosed, respectively, in Tettelin et al., Science, March 2000, 287: 1809 or WO 00/66791; Parkhill et al., Nature (March 2000) 404: 502; and Bentley et al., PLoS Genet., 3, e23 (2007).

Since the identification of these last sequences was made in the context of total sequencing of the genomes, an order number was attributed thereto. Thus, in Tettelin et al. (see above) or WO 00/66791, the sequences tpbB/TbpB of the strain MC58 are designated under the reference NMB 0460. In the rest of the text, the proteins of .N meningitidis may be designated without this making a limiting reference to the sequences of the strain MC58.

In the case of N. meningitidis, two major families of TbpB have been documented to date: isotype I characterized by a 1.8-kb tbpB gene and isotype II characterized by a 2.1-kb tbpB gene (EP 560 969 and EP 586 266). Isotype I is expressed in the ST-11 clonal complex and II in the clonal complexes ST-8, ST-18, ST-32 and ST-41/44 (Harrison et al., BMC Microbiol. 2008, 8: 66). The strains B16B6 (serogroup B) and FAM18 (serogroup C) are representatives of isotype I; the strains M982, BZ83 and 8680 are representatives of isotype II.

For the use for the purposes of the present invention, the lipidized TbpB is that of a strain of N. meningitidis of isotype I or of isotype II, preferably of isotype II. According to one particular embodiment, a composition according to the invention comprises the lipidized TbpB of a strain of isotype I and of a strain of isotype II. The lipidized TbpB of a strain of N. meningitidis of isotype I may be that of the strain B16B6; and the lipidized TbpB of a strain of N. meningitidis of isotype II may be that of the strain M982.

On account of its lipidized tail, it is expected that, in purified form, the lipidized and purified TbpB has a certain degree of insolubility under purely aqueous conditions. Consequently, it should be placed under conditions that promote its solubility. A person skilled in the art masters the techniques for making a lipoprotein soluble. It is possible, for example, to use a detergent during the purification of the lipoprotein, so as to obtain a preparation of a soluble purified lipoprotein in the presence of detergent. The amount of detergent remaining in the final preparation will be controlled such that it is just necessary to maintain the purified lipoprotein in soluble form. Alternatively, it is possible to completely remove the detergent used during the purification and then to add another product which also has the capacity of maintaining the purified lipoprotein in soluble form.

When the LOS is formulated as liposomes, the TbpBs may be incorporated with the LOS into liposomes or alternatively as a simple mixture with LOS liposomes (LOS formulated as liposomes): this latter embodiment is, however, preferred.

When the LOS and the lipidized TbpB are incorporated together in liposomes (proteo-liposomes), the liposomes that are useful for this purpose are the same as those described previously for the formulation of the LOS alone. One means for successfully preparing this formulation consists in formulating the LOS and the lipidized TbpB together in liposomes, for example by reconstituting a lipid film in aqueous medium in the presence of LOS and lipidized TbpB, especially in:
a lipid:LOS mole ratio from 100 to 500, advantageously from 100 to 400; preferably from 200 to 300; most particularly preferably about 250; and/or
an LOS:lipidized TbpB mole ratio from 10⁻² to 10³, advantageously from 10⁻¹ to 10²; preferably from 1 to 50; most particularly preferably from 15 to 30, e.g. about 20.

After the incorporation of the LOS and the lipidized TbpB into liposomes, a mixture formed from ad hoc liposomes (proteoliposomes), LOS and/or lipidized TbpB in free form may commonly be obtained. Advantageously, the liposomes are then purified so as to eliminate the LOS in free form. Once the free LOS has been removed, the mixture may be used as obtained for vaccine purposes, or alternatively the liposomes may be further purified so as to eliminate the free lipidized TbpB. Once the liposomes have been completely purified, it may be envisioned to add free lipidized TbpB, especially in a defined amount.

A vaccine/pharmaceutical composition according to the invention is especially useful for treating or preventing an infection caused by N. meningitidis, such as meningitis caused by N. meningitidis, meningococcemias and complications that may derive therefrom such as purpura fulminans and septic shock; and also arthritis and pericarditis caused by N. meningitidis.

It may be manufactured in a conventional manner. In particular, a therapeutically or prophylactically effective amount of the essential constituents of the vaccine, LOS and TpbB, is combined with a pharmaceutically acceptable support or diluent. Advantageously, it may also comprise a pharmaceutically acceptable adjuvant.

For use in a composition according to the invention, the LOS(s) is (are) advantageously formulated as liposomes.

The amounts of LOS and of rTpbB per vaccine dose which are effective from an immunogenic, prophylactic or therapeutic point of view, depend on certain parameters that include the individual treated (adult, adolescent, child or infant), the route of administration and the administration frequency.

Thus, the amount of LOS per dose may be between 5 and 500 Mg, advantageously between 10 and 200 Mg, preferably between 20 and 100 Mg, most preferably between 20 and 80 Mg or between 20 and 60 Mg, limits included.

The amount of lipidized Tpb B per dose may be between 5 and 500 μg, advantageously between 10 and 200 μg, preferably between 20 and 100 μg, entirely preferably between 20 and 80 μg or between 20 and 60 μg, limits included.

In the vaccine according to the invention, the mole ratio LOS: lipidized Tpb B is from 10⁻² to 10³, advantageously from 10⁻¹ to 10²; preferably from 1 to 50; most particularly preferably from 15 to 30 or about 20. By way of example, it is indicated that the mole ratio LOS: lipidized TpbB may typically be about 20 or 25, depending on whether the TpbB isotype is I or II.

The term “dose” employed above should be understood to denote a volume of vaccine administered to an individual in one go—i.e. at a time T. Conventional doses are of the order of a milliliter, for example 0.5, 1 or 1.5 ml; the definitive choice depending on certain parameters, and in particular on the age and the status of the recipient. An individual can receive a dose divided up into injections at several injection sites on the same day. The dose may be a single dose or, if necessary, the individual may also receive several doses a certain time apart—it being possible for this time apart to be determined by those skilled in the art.

A composition according to the invention may be administered by any conventional route in use in the prior art, e.g. in the vaccines field, in particular enterally or parenterally. The administration may be carried out as a single dose or as repeated doses a certain time apart. The route of administration varies as a function of various parameters, for example of the individual treated (condition, age, etc.).

Finally, a subject of the invention is also:
a method for inducing in a mammal, for example a human, an immune response against N. meningitidis, according to which an immunogenically effective amount of a composition according to the invention is administered to the mammal so as to induce an immune response, in particular a protective immune response against N. meningitidis; and
a method for prevention and/or treatment of an infection caused by N. meningitidis, according to which a prophylactically or therapeutically effective amount of a composition according to the invention is administered to an individual in need of such a treatment.

### Experimental Data

### A Experimental Data Relating to the Strains Derived from N. Meningitidis C708

### 1. Materials & Methods

### 1.1 Transformation of the Strain C708

The strain C708 is cultured in BHI (Brain Heart Infusion) agar medium at 37° C. under an atmosphere containing 10% CO₂. The bacterial lawn is harvested in BHI liquid medium complemented with 5 mM MgCl₂ to obtain a bacterial suspension at 10⁹ cfu/ml (cfu: colony-forming unit).

To 900 μl of the BHI liquid medium+5 mM MgCl₂ are added 10 μg of DNA necessary for the transformation (linearized plasmid); followed by 100 μl of the bacterial suspension (10⁸ microorganisms). The transformation medium is incubated for 30 minutes at 37° C., 10% CO₂.

500 μl of this preparation (i.e. about 5.10⁷ cfu) serve to inoculate 4.5 ml of BHI+5 mM MgCl₂. The bacteria are left to regenerate for 2 hours at 37° C., 10% CO₂. Next, starting with this suspension, dilutions of BHI+5 mM MgCl₂ are made. 300 μl of a dilution containing about 30 000 cfu are plated out on a 140 mm agar BHI dish. The dishes are placed at 37° C., 10% CO₂ for at least 20 hours.

### 1.2. Blotting of the Transformant Colonies

The colonies are transferred onto 137 mm Hybond-XL membrane (GE Healthcare; #RPN 137S). The microorganisms deposited on the membrane are lysed in denaturing buffer (0.5 M NaOH, 1.5 M NaCl). The membranes are washed with neutralizing buffer (0.5 M Tris, 1.5 M NaCl, pH 7.5); transferred into SSC 2× medium; and then dried. The DNA is bound by incubation for 2 hours at 80° C.

1.3. Detection of the Transformants by Hybridization with a Probe Labeled with ³³P dCTP

Labeling of the probe is obtained by PCR amplification using the Ready-to-Go PCR Beads kit (GE Healthcare); the labeled probe is then purified on a ProbeQuant G50 Microcolumn column (GE Healthcare).

The membranes to be hybridized are placed in threes in 50 ml of Rapid Hyb buffer (GE Healthcare) for 15 minutes at 65° C., with slow stirring, for prehybridization. The probe labeled and denatured beforehand for 2 minutes at 95° C. is added to the membranes. The final probe concentration is 5 ng/ml. Hybridization is allowed to continue for 2 hours at 65° C., with slow stirring.

The membranes are then subjected to successive washes by working as follows:
in low stringency buffer (2×SSC, 0.1% SDS (weight/vol)) 15 minutes at ambient temperature with slow stirring;
in medium stringency buffer (1×SSC, 0.1% SDS (weight/vol)) 20 minutes at 65° C. with slow stirring; and
low stringency buffer (0.1×SSC, 0.1% SDS (weight/vol)) 45 minutes at 65° C. with slow stirring.

Once dried, the membranes are revealed by autoradiography (Biomax MR film).

1.4. Detection of the Transformants by Hybridization with an Oligonucleotide Labeled with [γ³²P] ATP

Labeling of the 5′ end of the oligonucleotide is performed in the following reaction medium (the amounts indicated are those corresponding to the hybridization of an amount of oligonucleotide necessary for the hybridization of 3 membranes in a dish):

| | | | |
|---|---|---|---|
| free 5′-OH oligonucleotide | 3 | μl max | i.e. 10 pmol |
| 10X phosphorylation buffer | 1 | μl | i.e. 1 X |
| [γ-³²P] ATP 10 mCi/ml | 5 | μl | i.e. 50 μCi |
| T4 kinase (10 U/μl) | 1 | μl | i.e. 10 U |
| H₂O | qs 10 | μl | |

The reaction medium is incubated at 37° C. for 30 minutes. Next, the T4 kinase is inactivated by heating for 10 minutes at 70° C.

The membranes to be hybridized are placed in threes in 60 ml of Rapid Hyb buffer (GE Healthcare) for 15 minutes at 48° C., with slow stirring, for prehybridization. The prehybridization buffer is removed and replaced with 50 ml of hybridization buffer as follows: 5×SSC, 5×Denhardt's solution, 0.5% SDS (weight/vol.) and 100 μg/ml of salmon sperm DNA at 10 mg/ml sonicated and denatured for 5 minutes at 100° C.

The labeled oligonucleotide (10 μl) is added to the membranes. The hybridization is allowed to continue overnight at a temperature 5° C. below the Tm of the oligonucleotide, with gentle stirring.

The membranes are then subjected to successive washes by working in the order as follows:
in low stringency buffer (2×SSC, 0.1% SDS (weight/vol) 5 minutes at the Tm of the oligonucleotide −5° C., with slow stirring;
in medium stringency buffer (1×SSC, 0.1% SDS (weight/vol) 15 minutes at the Tm of the oligonucleotide −5° C., with slow stirring; and
in low stringency buffer (0.1×SSC, 0.1% SDS (weight/vol) 10 minutes at the Tm of the oligonucleotide −5° C., with slow stirring.

Once dried, the membranes are revealed by autoradiography (Biomax MR film).

2. Construction of a Strain of N. Meningitidis Expressing an Los Whose a Chain is that of an Los of Immunotype L6 and Comprising in Each of the Positions O3 and O6 of the heptose II (hep II) Residue of the Inner Core a Phospho-Ethanolamine (PEA) Substituent

The starting strain used is the strain of N. meningitidis C708 of serogroup A and of immunotype L6 having, inter alia, the following characteristics:
an active lgtA gene (gene switched “ON”);
an lgtB gene—(non-functional gene);
an inactive lgtG gene (switched “OFF”);
a truncated lpt3 gene;
an active lpt6 gene; and
an active lot3 gene.

The strain C708 was filed on 11 Mar. 2008 at the Collection Nationale de Culture de Microorganisme, 25 rue du Dr Roux 75015 Paris, according to the terms of the treaty of Budapest. This strain bears the order number CNCM I-3942.

The strain C708 comprises a truncated lpt3 gene. To modify it such that the LOS bears a PEA substituent in position O3, it is chosen to replace by homologous recombination the truncated lpt3 gene with the complete (full-length) lpt3 gene of the strain of N. meningitidis FAM18 serogroup C (strain made available worldwide to research laboratories). The strain resulting therefrom will be referred to for greater convenience as C708 lpt3 FL.

2.1. PCR (Polymerase Chain Reaction) Amplification of the Full-Length (FL) lpt3 Gene of the Strain of N. Meningitidis FAM18

100 ng of genomic DNA of the strain FAM18 were used for amplification with Platinum® Taq DNA polymerase High Fidelity (Invitrogen, #11304-011).

The pair of primers is as follows (pair No. 1):

CG GAATTC GCC GTC TCA A ATG AAA AAA TCC CTT TTC GTT
CTC (Tm = 55.9° C.);
and
AA CTGCAG TCA TTG CGG ATA AAC ATA TTC CG
(Tm = 57.1° C.);
(the EcoRI and PstI sites are respectively
underlined).

The following mixture was used for amplification:

| Components | Volume | | Final concentration |
|---|---|---|---|
| 10X High Fidelity PCR buffer | 5 | μl | 1X |
| 10 mM dNTP mixture | 1 | μl | 0.2 mM of each |
| 50 mM MgSO₄ | 2 | μl | 2 mM |
| Mixture of primers (10 μM each) | 1 | μl | 0.2 μM of each |
| Genomic DNA | x | μl | 100 ng |
| Platinum ® Taq High Fidelity | 0.2 | μl | 1.0 unit |
| Nuclease-free water | qs 50 | μl final | Does not apply |

The thermocycler program is as follows:

| Initial denaturing: | | 94° C. for 30 seconds |
|---|---|---|
| 30 cycles of: | denaturing: | 94° C. for 30 seconds |
| | hybridization: | 55° C. for 30 seconds |
| | extension: | 68° C. for 1 minute/kb of PCR product. |

After the reaction, 1/10 of the PCR product was deposited on agarose gel for verification.

### 2.2. Construction of a Transformation Vector

The PCR product, on the one hand, and the plasmid pUC19, on the other hand, were subjected to double digestion with EcoRI and PstI for 2 hours at 37° C. 10 units of each enzyme per μg of DNA in REact2 buffer (Invitrogen) were used.

The PCR fragment was then inserted into the linearized pUC19 vector. The ligations were performed in a final volume of 20 μl with 50 ng of vector, 0.5 U of T4 DNA ligase (Invitrogen) and 1 μl of 10 mM ATP (Invitrogen) for 16 hours at 16° C. The ligase was then inactivated by heating for 10 minutes at 65° C.

The vector thus obtained was transferred via the electroporation technique into a strain of E. coli X1,1 blue MRF resistant to kanamycin and made electrocompetent. The parameters adopted for the electroporation are as follows: capacitance: 500 μFD; resistance: 200 ohms; voltage: 1700 volts.

Selection of the transformed clones was performed by plating out on 100 μg/ml ampicillin LB dishes. Authentification of 10 of the 50 positive clones was performed by PCR amplification. 100% of the clones had the expected profile. Finally, the lpt3 gene in a plasmid of one of these clones (plasmid pM1222) was verified by sequencing.

### 2.3. Transformation of the Strain C708 and Detection of the Homologous Recombination Event

### 2.3.1. Transformation

40 μg of pM1222 were digested with 400 U of EcoRI and 10 m were used to transform the strain C708 according to the method described in section A.1.1.

After transformation, the bacteria were plated out onto 17 140 mm Petri dishes at a theoretical concentration of 30 000 cfu per dish; i.e. 510 000 cfu (colony-forming units) and were then placed overnight at 37° C. The dishes were then placed at +4° C. for 30 minutes.

After 24 hours at 37° C., counting of the control dishes made it possible to estimate the number of cfu as 27 000 per dish for the mutant.

### 2.3.2. Clone Selection

The recombination event, i.e. the replacement of the lpt3 TR (truncated) gene with the lpt3 FL (full-length) gene, was detected after transferring the clones onto hybridization membrane and hybridizing with a probe labeled with ³³P dCTP according to the methods described in sections A.1.2. and A.1.3.

Selection of the positive clones was made by hybridization of the DNA bound to the membranes with a DNA probe labeled with ³³P dCTP corresponding to the truncated part of the lpt3 gene, which is thus present only in the recombinant clones.

### Preparation of the Probe

In order to obtain the 270-bp lpt3 probe, 10 ng of the plasmid pUClpt3 were used for PCR amplification with Platinum® Taq DNA polymerase High Fidelity (Invitrogen, #11304-011).

The pair of primers is as follows (pair No. 2):

CGC CGA ATA CTT TAT CTT GAG GC (Tm = 60.6° C.);
and
CTC GCC AAA GAG CAG GGC (Tm = 60.5° C.).

For amplification, the following mixture was used:

| Components | Volume | | Final concentration |
|---|---|---|---|
| 10X High Fidelity PCR buffer | 5 | μl | 1X |
| 10 mM dNTP mixture | 1 | μl | 0.2 mM of each |
| 50 mM MgSO₄ | 2 | μl | 2 mM |
| Mixture of primers (10 μM each) | 1 | μl | 0.2 μM of each |
| Plasmid pUC | x | μl | 100 ng |
| Platinum ® Taq High Fidelity | 0.2 | μl | 1.0 unit |
| Nuclease-free water | qs 50 | μl final | Does not apply |

The thermocycler program is as follows:

| Initial denaturing: | | 94° C. for 30 seconds |
|---|---|---|
| 30 cycles of: | Denaturing: | 94° C. for 30 seconds |
| | Hybridization: | 55° C. for 30 seconds |
| | Extension: | 68° C. for 45 seconds. |

After the reaction, 1/10 of the PCR products was deposited on agarose gel to ensure the specificity of the amplicon, and the PCR fragment was then purified using the QIAquick PCR Purification Kit (Qiagen, #28104).

### Hybridization and Revelation

The steps of labeling of the probe, hybridization, washing and revelation were performed as described in section A.1.3.

About 460 000 cfu were tested. After exposure with the BioMax MR films, the autoradiographs revealed 5 positive spots (C708 containing an lpt3 FL gene) each on a different membrane.

### Screening and Authentification of the Positive Clones

After locating on the Petri dish, part of the zone taken up around the positive clones was stored in freezing medium (M199 medium, 20% fetal calf serum, 10% glycerol) and the other part was used for the PCR authentification.

To do this, each of the samples collected was first taken up in 80 μl of BHI broth, so as to plate out 30 μl of this suspension, as a mini-lawn on a BHI dish.

The remaining volume was centrifuged for 5 minutes at 6000 rpm and the pellet was then taken up in 50 μl of nuclease-free water. The microorganisms were lysed for 5 minutes at 95° C. and the supernatant, which serves as the matrix for the PCR reaction, was collected after centrifugation.

For each collected sample corresponding to a positive spot and for the controls, PCR amplification with the pair of primers that served for the amplification of the 270 bp C708 lpt3 probe (pair No. 2) was performed with the Expand Long Template PCR kit (Roche) as described below.

| Components | Volume | | Final concentration |
|---|---|---|---|
| 10X ELT PCR buffer | 5 | μl | 1X |
| dNTP mixture (10 mM of each) | 2 | μl | 0.4 mM of each |
| mixture of primers (10 μM of each) | 1.5 | μl | 0.3 μM of each |
| DNA matrix | 40 | μl | Does not apply |
| Polymerase ELT | 0.75 | μl | 3.75 units |
| Nuclease-free water | qs 50 | μl | Does not apply |

The thermocycler program is as follows:

| Initial denaturing: | | 94° C. for 2 min |
|---|---|---|
| 10 cycles of: | denaturing: | 94° C. for 10 seconds |
| | hybridization: | 54° C. for 30 seconds |
| | extension: | 68° C. for 45 seconds |
| 20 cycles of: | denaturing: | 94° C. for 15 seconds |
| | hybridization: | 54° C. for 30 seconds |
| | extension: | 68° C. for 45 seconds + 20 sec/cycle |

| | |
|---|---|
| Final elongation: | 68° C. for 7 min |

After the reaction, 1/10 of the PCR products was deposited on agarose gel for verification. Four of the 5 clones had the expected profile. The frequency of production of a true positive clone was 1/115 000 cfu tested.

The following step consisted in isolating a pure clone. To do this, one of the heterogeneous positive clones was plated out as isolated cfus and several of these cfus (40) were analyzed by PCR, with the pairs of primers 1 or 2.

Each cfu was resuspended in 100 μl of nuclease-free water, 30 μl were deposited on a BHI dish and the remaining 70 μl were lysed for 5 minutes at 95° C., and the supernatant, which serves as the matrix for the PCR reaction, was collected after centrifugation.

The PCRs were performed with Platinum® Taq High Fidelity (Invitrogen) as already described for the amplification of the lpt3 probe. The hybridization temperature was 54° C.

After the reaction, 1/10 of the PCR products was deposited on agarose gel for verification. Five of the 40 clones proved to be pure clones.

The mini-lawn of pure clones was taken up in freezing medium, divided into 100 μl aliquots and stored at −70° C. The purity and the identity of this freezing material were validated.

3. Construction of a Strain of N. Meningitidis Expressing an Los Whose a Chain is that of an Los of Immunotype L6 and Comprising Only in Position O3 of the Heptose II (Hep II) Residue of the Inner Core a Phosphoethanolamine (PEA) Substituent

The starting strain used is the strain of N. meningitidis C708 lpt3 FL obtained as described previously. The object is to inactivate the lpt6 gene of this strain by deletion of a central part of the gene.

### 3.1. PCR (Polymerase Chain Reaction) Amplification of the Full-Length Lpt6 Gene of the Strain of N. Meningitidis 22491 of Serogroup a (Gene NMA 0408)

100 ng of genomic DNA of the strain Z2491 (strain made available worldwise to research laboratories) were used for amplification with Platinum® Taq DNA polymerase High Fidelity (Invitrogen, #11304-011).

The pair of primers is as follows (pair No. 3):

CG GAATTC GCC GTC TCA A GGT TGC CTA TGT TTT CCT GTT
TTT G (Tm = 59.7° C.);
and
AA CTGCAG CTA ACG GGC AAT TTT CAA AAC GTC
(Tm = 59.3° C.);
(the EcoRI and PstI sites are respectively
underlined).

For amplification the following mixture was used:

| Components | Volume | | Final concentration |
|---|---|---|---|
| 10X High Fidelity PCR buffer | 5 | μl | 1X |
| 10 mM dNTP mixture | 1 | μl | 0.2 mM of each |
| 50 mM MgSO₄ | 2 | μl | 2 mM |
| Mixture of primers (10 μM each) | 1 | μl | 0.2 μM of each |
| Genomic DNA | x | μl | 100 ng |
| Platinum ® Taq High Fidelity | 0.2 | μl | 1.0 unit |
| Nuclease-free water | qs 50 | μl final | Does not apply |

The thermocycler program is as follows:

| Initial denaturing: | | 94° C. for 30 seconds |
|---|---|---|
| 30 cycles of: | denaturing: | 94° C. for 30 seconds |
| | hybridization: | 55° C. for 30 seconds |
| | extension: | 68° C. for 1 minute/kb of PCR product. |

After the reaction, 1/10 of the PCR product was deposited on agarose gel for verification.

3.2. Construction of the Vector pM1223 (pUC19 lpt6 FL)

The PCR product, on the one hand, and the plasmid pUC19, on the other hand, were subjected to double digestion with EcoRI and PstI for 2 hours at 37° C. 10 units of each enzyme per μg of DNA were used in the buffer REact2 (Invitrogen).

The PCR fragment was then inserted into the linearized pUC19 vector. Ligations were performed on a final volume of 20 μl with 50 ng of vector, 0.5 U of T4 DNA ligase (Invitrogen) and 1 μl of 10 mM ATP (Invitrogen) for 16 hours at 16° C. The ligase was then inactivated by heating for 10 minutes at 65° C.

The vector thus obtained was transferred via the electroporation technique into a strain of E. coli XL1 blue MRF resistant to kanamycin and made electrocompetent. The parameters adopted for the electroporation are as follows: capacitance: 5000D; resistance: 200 ohms; voltage: 1700 volts.

Selection of the transformed clones was performed by plating out onto 100 μg/ml ampicillin LB dishes. Authentification of the positive clones (presence of an lpt6 FL gene) was performed by NdeI enzymatic digestion after extraction of the DNA by miniprep. Out of 20 clones analyzed, 6 had the expected profile. The recombinant plasmid of the selected clone was named pM1223.

3.3. Deletion of the Central Part of the lpt6 Gene Originating from the Strain Z2491

Construction of a Transformation Vector

With the Expand Long Template PCR kit (Roche), a reverse PCR was performed using the plasmid pM1223 with the aid of the following pair of primers (pair No. 4):

CG GGATCC CAT CGA CAC GAA CGC CGC (Tm = 60.5°);
and
CG GGATCC CCG CGC TTA ACG ACT ACA TC (Tm = 59.4°);
(the BamHI sites are underlined).

This makes it possible to reamplify the plasmid while deleting the part that it is desired to remove (808 bp).

The following mixture was used for amplification:

| Components | Volume | | Final concentration |
|---|---|---|---|
| 10X ELT PCR buffer | 5 | μl | 1X |
| dNTP mixture (10 mM of each) | 2 | μl | 0.4 mM of each |
| Mixture of primers (10 μM of each) | 1.5 | μl | 0.3 μM of each |
| DNA matrix (pM1223) | 40 | μl | Does not apply |
| Polymerase ELT | 0.75 | μl | 3.75 units |
| Nuclease-free water | qs 50 | μl | Does not apply |

The thermocycler program is as follows:

| Initial denaturing: | | 94° C. for 2 minutes |
|---|---|---|
| 10 cycles of: | denaturing: | 94° C. for 10 seconds |
| | hybridization: | 54° C. for 30 seconds |
| | extension: | 68° C. for 3 minutes |
| 20 cycles of: | denaturing: | 94° C. for 15 seconds |
| | hybridization: | 54° C. for 30 seconds |
| | extension: | 68° C. for 3 minutes + 20 sec/cycle |

| | |
|---|---|
| Final elongation: | 68° C. for 7 minutes |

After the reaction, 1/10 of the PCR products was deposited on agarose gel.

After purification on a QiaQuick column, the PCR product was digested with BamHI at a rate of 10 U of enzyme per μg of DNA. Once digested, it was purified by electro-elution and then extracted with phenol-chloroform.

Self-ligation of the vector was performed in a final volume of 20 μl with 0.5 U of T4 DNA ligase (Invitrogen) and 1 μl of 10 mM ATP (Invitrogen) for 16 hours at 16° C. The ligase was then inactivated by heating for 10 minutes at 65° C.

The final step consisted in transferring the vector thus ligated into a strain of Escherichia coli as described for pM1222. Authentification of the positive clones was performed by NdeI-PstI enzymatic digestion after extraction of the DNA by miniprep. Out of the 4 clones analyzed, 100% had the expected profile.

The recombinant plasmid of the selected positive clone was named pM1224, and this clone was stored in glycerol at −70° C. The presence in the plasmid pM1224 of an lgt6 gene with its central part deleted was confirmed by sequencing.

3.4. Transformation of the Strain C708 lpt3 FL and Detection of the Homologous Recombination Event

### Transformation

10 μg of plasmid pM1224 were linearized with EcoRI at a rate of 10 units of enzyme per μg of plasmid to be digested in the appropriate buffer for 2 hours at 37° C.

The transformation in the strain C708 was performed according to the technique described in section A.1.1.

After transformation, the bacteria were plated out on 16 140 min Petri dishes at a theoretical concentration of 50 000 cfu per dish; i.e. 800 000 cfu. The dishes were placed overnight at 37° C. and then placed for 30 minutes atà+4° C.

### Clone Selection: Preparation of the Probe, Hybridization and Revelation

The recombination event was detected after colony blotting and hybridization with an oligonucleotide labeled with γ³²P dATP.

The recombination event, i.e. the replacement of the lpt6 FL gene with the lpt6 TR gene, was detected after transferring the clones onto membrane and hybridization with a labeled probe according to the methods described in sections A.1.2. and A.1.4.

The clones transferred onto membranes were subjected to lysis and washing steps. The DNA is bound to the membranes by placing them for 2 hours at 80° C.

Selection of the positive clones was performed by hybridization of the DNA bound to Hybond N+membranes with a radioactive oligonucleotide whose sequence overlaps the two recombigenic ends. This is the following oligonucleotide: GTC GAT GGG ATC CCC GCG CTT AAC G (Tm=69.5° C.).

About 840 000 cfu were tested. After exposure with BioMax MR films, the autoradiographs revealed 16 positive spots (C708 containing an lpt6 TR gene) divided among 9 different membranes.

### Screening and Authentification of the Positive Clones

For each of the 16 positive spots, after detection on the Petri dish, part of the zone collected around the positive clones was stored in freezing medium (M199, 20% FCS, 10% glycerol) and the other part was used for the PCR authentification.

To do this, the collected samples were first taken up in 80 μl of BHI broth, so as to plate out, as a mini-lawn on a BHI dish, 30 μl of each suspension.

The remaining volume was centrifuged for 5 minutes at 6000 rpm, and the pellet was then taken up in 50 μl of nuclease-free water. The microorganisms were lysed for 5 minutes at 95° C. and the supernatant, which serves as the PCR reaction matrix, was collected after centrifugation.

For each collected sample corresponding to a positive spot, a PCR amplification PCR was performed with the Platinum® Taq High Fidelity kit (Invitrogen) and the following pair of primers (pair No. 5)

CCG ACT GGC GGA ATT GGG (TM = 60.5° C.);
and
CCC ATT TCT TCC TGA CGG AC (Tm = 59.4° C.).

The following mixture was used for amplification:

| Components | Volume | | Final concentration |
|---|---|---|---|
| 10X High Fidelity PCR buffer | 5 | μl | 1X |
| 10 mM dNTP mixture | 1 | μl | 0.2 mM of each |
| 50 mM MgSO₄ | 2 | μl | 2 mM |
| Mixture of primers (10 μM each) | 1 | μl | 0.2 μM of each |
| DNA matrix | x | μl | 100 ng |
| Platinum ® Taq High Fidelity | 0.2 | μl | 1.0 unit |
| Nuclease-free water | qs 50 | μl final | Does not apply |

The thermocycler program is as follows:

| Initial denaturing: | | 94° C. for 1 minute |
|---|---|---|
| 30 cycles of: | denaturing: | 94° C. for 30 seconds |
| | hybridization: | 55° C. for 30 seconds |
| | extension: | 68° C. for 50 seconds. |

After the reaction, 1/10 of the PCR product was deposited on agarose gel, for verification. Two candidates out of 16 proved to be true positives: i.e. a frequency of production of one true positive clone per 425 000 cfu tested.

The following step consisted in isolating a pure clone. To do this, one of the 2 heterogeneous positive clones was plated out as isolated cfus and several of these cfus (24) were analyzed by PCR, with the pair of primers No. 5.

Each cfu was resuspended in 50 μl of nuclease-free water, 20 μl were deposited on a BHI dish and the remaining 30 μl were lysed for 5 minutes at 95° C. and the supernatant, which serves as PCR reaction matrix, was collected after centrifugation.

The PCRs were performed with the Platinum® Taq High Fidelity kit (Invitrogen) as already described for the screening of the collected samples of the positive spots.

After the reaction, 1/5 of the PCR products was deposited on agarose gel for verification. Only one clone out of 24 proved to be a true positive.

The mini-lawn of the pure positive clone was taken up in freezing medium, divided into 100 μl aliquots and stored at −70° C. The purity and identity of this frozen material were confirmed.

4. Construction of a Strain of N. Meningitidis Expressing an Los Whose a Chain is that of an Los of L8 Immunotype and Comprising Only in Position O3 of the Heptose II (Hep II) Residue of the Inner Core a Phosphoethanolamine (PEA) Substituent

The starting strain used is the strain of N. meningitidis C708 lpt3 FL lpt6 TR obtained as described previously in section A.3. The objective is to inactivate the lgtA gene of this strain by deletion of a central part of the gene.

### 4.1. PCR (Polymerase Chain Reaction) Amplification of the Full-Length lgt a Gene of the Strain of N. Meningitidis MC58 of Serogroup B (Gene NMB 1929)

100 ng of genome DNA of the strain MC58 (strain available worldwide in research laboratories) were used for amplification with Platinum® Taq DNA polymerase High Fidelity (Invitrogen, #11304-011).

The pair of primers is as follows:

CG GAATTC GCC GTC TCA A ATG CCG TCT GAA GCC TTC AG
(Tm = 59.4° C.);
and
AA CTGCAG AAC GGT TTT TCA GCA ATC GGT GC
(Tm = 60.6° C.).
(the EcoRI and PstI sites, respectively, are
underlined).

For amplification, the following mixture was used:

| Components | Volume | | Final concentration |
|---|---|---|---|
| 10X High Fidelity PCR buffer | 5 | μl | 1X |
| 10 mM mixture of dNTP | 1 | μl | 0.2 mM of each |
| 50 mM MgSO₄ | 2 | μl | 2 mM |
| Mixture of primers (10 μM each) | 1 | μl | 0.2 μM of each |
| Genome DNA | x | μl | 100 ng |
| Platinum ® Taq High Fidelity | 0.2 | μl | 1.0 unit |
| Nuclease-free water | for 50 | μl final | not applicable |

The thermocycler program is as follows:

| | | |
|---|---|---|
| Initial denature: | | 94° C. for 30 seconds |
| 30 cycles of: | denature: | 94° C. for 30 seconds |
| | hybridization: | 55° C. for 30 seconds |
| | extension: | 68° C. for 1 minute/kb of PCR product. |

After the reaction, 1/10 of PCR product was deposited on agarose gel for verification.

4.2. Construction of the Vector pUC19 lgtA FL

The PCR product obtained in 4.1., on the one hand, and the plasmid pUC19, on the other hand, were subjected to a double digestion with EcoRI and PstI for 2 hours at 37° C. 10 units of each enzyme were used per μg of DNA in the REact2 buffer (Invitrogen).

The PCR fragment was then inserted into the linearized pUC19 vector. Ligations were performed in a final volume of 20 μl with 50 ng of vector, 0.5 U of T4 DNA ligase (Invitrogen) and 1 μl of 10 mM ATP (Invitrogen) for 16 hours at 16° C. The ligase was then inactivated by heating for 10 minutes at 65° C.

The vector thus obtained was transferred via the electroporation technique into the kanamycin-resistant and electrocompetent-rendered strain of E. coli XL1 blue MRF. The following parameters were applied for the electroporation: capacitance: 500 μFD; resistance: 200 ohms; voltage: 1700 volts.

Selection of the transformed clones was made by plating out onto LB ampicillin dishes 100 μg/ml. Authentification of the positive clones (presence of an lgtA FL gene) was made by enzymatic digestion after miniprep extraction of the DNA. 1/5 of the analyzed clones had the expected enzymatic digestion profile.

### 4.3. PCR (Polymerase Chain Reaction) Amplification of the Erythromycin (Erm)-Resistant Gene

PCR amplification of the erythromycin (erm) cassette starting with pMGC10 was performed with primers enabling integration of the BamHI-XbaI restriction sites.

The pair of primers used is as follows:

CG GGATCC GGA AGG CCC GAG CGC AGA AGT
(Tm: 65.7° C.);
and
GC TCTAGA CAA CTT ACT TCT GAC AAC GAT CGG
(Tm: 61° C.)

For amplification, the following mixture was prepared:

| Components | Volume | | Final concentration |
|---|---|---|---|
| Pfu turbo 10X buffer | 5 | μl | 1X |
| 10 mM dNTP mixture | 0.4 | μl | 0.2 mM of each |
| Mixture of primers (10 μM each) | 1 | μl | 0.2 |
| pMGC10 | x | μl | 10 ng |
| Pfu turbo (Stratagene) | 1 | μl | 2.5 units |
| Nuclease free water | to 50 | μl | not applicable |

The thermocycler program was as follows:
Initial denature: 95° C. for 2 minutes
30 cycles of:
Denature: 95° C. for 30 seconds
Anneal: 55° C. for 30 seconds
Extend: 72° C. for 1 minute
Final elongation: 72° C. for 10 nm

After the reaction, 1/10 of the PCR products are deposited onto agarose gel.

4.4. Construction of the Plasmid pUC19 lgtA TR (Deletion of the Central Part of the lgtA Gene by Inverse PCR)

With the kit Expand Long Template PCR (Roche), inverse PCR was performed starting with the plasmid pUC19 lgtA FL, obtained in A.4.2, for the twofold purpose of removing the central part of the gene and creating two restriction sites at the ends (BamHI and XbaI). The pair of primers below was used:

CG GGATCC GCC AAT TCA TCC AGC CCG ATG
(Tm = 61.8° C.);
and
CG TCTAGA CCC GGT TCG ACA GCC TTG
(Tm = 60.5° C.);

This makes it possible to reamplify the plasmid by deleting the part that it is desired to remove.

For amplification, the following mixture was prepared:

| Components | Volume | | Final concentration |
|---|---|---|---|
| 10X ELT PCR buffer | 5 | μl | 1X |
| Mixture of dNTP (10 mM of each) | 2 | μl | 0.4 mM of each |
| Mixture of primers (10 μM of each) | 1.5 | μl | 0.3 μM of each |
| DNA matrix (10 ng of pUC19 lgtA | | | not applicable |
| FL) | | | |
| Polymerase ELT | 0.75 | μl | 3.75 units |
| Nuclease-free water | for 50 | μl | not applicable |

The thermocycler program is as follows:

| | | |
|---|---|---|
| Initial denature: | | 94° C. for 2 minutes |
| 10 cycles of: | Denature: | 94° C. for 10 seconds |
| | Hybridization: | 55° C. for 30 seconds |
| | Extension: | 68° C. for 1 min per kbs |
| 20 cycles of: | Denature: | 94° C. for 15 seconds |
| | Hybridization: | 55° C. for 30 seconds |
| | Extension: | 68° C. for 1 min per kbs + 20 sec/cycle |

| | |
|---|---|
| Final elongation: | 68° C. for 7 min |

After the reaction, 1/10 of the PCR products was deposited on agarose gel for verification of the size (3.2.kbs). The PCR product was purified on a QiaQuick column.

The final step consisted in transferring the vector into the kanamycin-resistant and electrocompetent-rendered strain of E. coli XL1 blue MRF. Authentification of the positive clones (lgtA deleted from its central part) was performed by enzymatic digestion after miniprep extraction of the DNA.

4.5. Construction of the Plasmid pUC19 lgtA Erm

The PCR erm product, on the one hand, and the plasmid pUC19 lgtA TR obtained from inverse PCR, on the other hand, were each subjected to a double digestion with BamHI and XbaI under the following conditions:

2 μg of DNA were placed in contact with 20 units of XbaI in buffer 2 (InVitrogen) in 60 μl for 2 hours at 37° C. Next, XbaI was inactivated for 10 minutes at 65° C. 7 μl of 1M NaCl, 20 units of BamHI and 1 μl of buffer 2 were then added. Digestion was continued for 2 hours at 37° C.

The digestion products were then deposited in their entirety on a 0.8% agarose gel and, after migration, the bands were chopped out for electroelution (that of the plasmid is at 3.2 kbs).

After purification, the linearized plasmid and the digested PCR erm product were ligated together as described previously. The ligation product was used to transform as described previously the kanamycin-resistant and electrocompetent-rendered strain of E. coli XL1 Blue MRF. The recombinant clones were analyzed by enzymatic digestion. 4/11 of the analyzed clones had the expected enzymatic digestion profile.

4.6. Transformation of the Strain C708 lpt3 FL lpt6 TR and Detection of the Homologous Recombination Event

10 μg of plasmid pUC19 lgtA erm were linearized with EcoRI at a rate of 10 units of enzyme per μg of plasmid to be digested in the appropriate buffer for 2 hours at 37° C.

Transformation of the strain C708 lpt3 FL lpt6 TR was performed by following the technique described in section A.1.1.

After transformation, 1.24×10⁸ bacteria were plated out on BHI medium+erythromycin at 2 μg/mL. Incubation was continued overnight at 37° C. The observed transformation frequency is 1/2.5×10⁶.

### B. Experimental Data Relating to the Vaccine Compositions

1. Preparation of the Lipidated rTbpB

In the interest of simplifying the language, the term “rTbpB” or “TbpB” will subsequently be simply indicated.

### 1.1. Production

### Strains Expressing the Lipid-Containing TbpB M982 or B16B6

The expression strains are the E. coli BL21 strains containing the pTG9219/pTG9216 plasmid respectively. These plasmids contains in particular a kanamycin-selectable marker and the polynucleotide encoding the rTbpB from the N. meningitidis M982 strain (pTG9219) or B16B6 (pTG9216) (the sequences are as described in patent EP 586 266), fused to the E. coli R1pB (real lipoprotein B) signal sequence and placed under the control of the arabinose promoter (araB).

### Culturing

Three frozen samples of the E. coli BL21/pTG9219 or BL21/pTG9216 strain (each 1 ml) are used to inoculate 3 liters of LB (Luria Broth) medium divided up in Erlenmeyer flasks. The incubation is continued for 15 to 18 h at 37° C.

This preculture is used to inoculate a fermenter containing TGM16 medium (9 g/L yeast extract, 0.795 g/L K₂SO₄, 3.15 g/L K₂HPO₄, 0.75 g/L NaCl, 0.005 g/L CaCl₂2H₂O, 0.021 g/L FeCl₃.6H₂O, 0.69 g/L MgSO₄.7H₂O, 37.5 g/L salt-free casein acid hydrolysate) supplemented with 20 g/L glycerol, in a proportion of 10% (vol./vol).

The culturing is continued at 37° C. with shaking, at a pressure of 100 mbar and with an air feed of 1 L/min/L of culture, while readjusting, over time, the glycerol concentration to 20 g/L (e.g. at OD₆₀₀ of 15±2). When the OD₆₀₀ is between 21 and 27, the rTbpB expression is induced by adding arabinose so as to obtain a final concentration of 10 g/L. After one hour of induction, the culture is stopped by cooling to around 10° C.

The bacterial pellets are recovered by centrifugation and stored in the cold.

### 1.2. Purification

Extraction of Membranes Containing the rTbpB

### LOS Extraction

A bacterial pellet equivalent to one liter of culture (approximately 72 g of microorganisms, wet weight) is thawed at a temperature of 20° C. +/−5° C. The thawed (or partially thawed) microorganisms are resuspended with 800 ml of a solution, at ambient temperature, of 50 mM Tris HCl, 5 mM EDTA, pH 8.0. 9 protease inhibitor tablets (7 Complete Mini, EDTA free tablets; ROCHE ref. 11836170001+two Complete, EDTA free tablets; ROCHE ref. 11836170001) are immediately added. Since some of the microorganisms lyze spontaneously, 4 μl of benzonase (1 IU of DNAse activity/ml final concentration; Merck ref. K32475095) are also added. The incubation is continued at +4° C. for 45 minutes with magnetic stirring after homogenization with a Turrax (15 sec.).

4 ml of 1M MgCl₂ are then added so as to be at a final concentration of 5 mM. The magnetic stirring is continued for 10 minutes. Centrifugation at 15 000 g for 45 minutes makes it possible to harvest the pellet (pellet P1; versus supernatant S1) containing the rTbpB protein.

A second extraction is carried out: homogenization with a Turrax in 800 ml of the 50 mM Tris HCl buffer containing 5 mM EDTA, pH 8.0, and stirring for 30 min. MgCl₂ (8 ml of a molar solution) is added. The incubation is continued for 10 minutes. The suspension is centrifuged at 15 000 g for 1 hour 30.

### Bacterial Lysis

The pellet is resuspended with 1400 ml of 50 mM Tris HCl supplemented with 4 protease inhibitor tablets with 8 μl of benzonase. The solution is homogenized with a Turrax for 15 seconds. The lysis is carried out at +4° C. for 30 minutes through the addition of 14 ml (10 mg/ml final concentration) of lysozyme at 100 mg/ml in 25 mM Na acetate, 50% glycerol.

The suspension is centrifuged at 30 000 g for 30 minutes (pellet P2 containing the protein; versus supernatant S2 containing the contaminants of rTbpB). The pellet containing the membranes can be frozen at this stage.

### Washing of Membrane Fragments

The lysis pellet P2 is taken up in 50 mM Tris HCl (1100 ml). After homogenization, (Turrax 15 seconds), it is washed for one hour at +4° C. A centrifugation is carried out as previously at 30 000 g for 30 minutes. The pellet (P3; versus supernatant S3) is frozen at −45° C. 50 mM Tris HCl buffer makes it possible to remove a small amount of protein (supernatant S3) and solubilizes only very little rTbpB.

The pellet P3 is taken up in 50 mM Tris HCl buffer containing 8M urea, pH 8.0 (800 ml). This buffer makes it possible to remove a part of the contaminating proteins without solubilizing the membranes containing the rTbpB. After homogenization (without using a Turrax), the solution is then stirred for one hour at +4° C. A centrifugation is carried out as previously at 30 000 g for 30 minutes, which makes it possible to obtain a membrane pellet which can be frozen.

### Membrane Solubilization

The thawed membrane pellet is solubilized with 780 ml of 50 mM Tris HCl buffer containing 6 mM EDTA, 2M urea and 4% elugent, at pH 7.5. The presence of the detergent at 4% and of the 2M urea makes it possible to solubilize the pellet. The solution is stirred at +4° C. overnight (minimum 16 h). Centrifugation of the solution at 30 000 g (1 hour at +4° C.) leaves only a small pellet (P4) containing a few impurities. The supernatant S4 containing the rTbpB protein is recovered for loading on a first cation exchange column (QS I).

### Purification by Anion Exchange Chromatography on Q Sepharose at pH 7.5

Two successive chromatographies are carried out, the product of the first chromatography is collected and then subsequently loaded, after a dialysis step, on a second chromatography column which uses different conditions (absence of EDTA).

### 1^{st} Chromatography, in the Presence of EDTA (Chromatography Qs I)

A column of 600 ml (K50, diameter 20 cm²) of Q Sepharose Fast Flow gel (ref. 17-0510-01 GE Healthcare) is mounted, tamped in equilibration buffer, 50 mM Tris HCl containing 6 mM EDTA, 2M urea and 1% elugent, at pH 7.5, at the flow rate of 8 ml/minute.

The supernatant S4 (approximately 845 ml) is loaded at the flow rate of 6 ml/minute. The direct eluate (part which does not attach to the column during loading of the sample) contains the protein of interest, rTbpB. This eluate (1150 ml) is taken and then dialyzed at +4° C. (for 6 days) against 6 liters of 50 mM Tris HCl buffer containing 2M urea and 1% elugent, pH 7.5, in order to reduce the EDTA concentration to 1 mM and to remove the NaCl.

2^{nd} Chromatography (QS II), without EDTA

A K50 column of 490 ml of new Q Sepharose Fast Flow gel is equilibrated in 50 mM Tris HCl buffer containing 2M urea and 1% elugent, pH 7.5.

The dialyzed solution (1080 ml) is loaded on the column (flow rate 6 ml/minute); then 5 saline elution steps in this same buffer are carried out: 20 mM, 50 mM, 100 mM, 250 mM and 1M NaCl (working flow rate 6 ml/minute). The rTbpB protein is eluted from the column at two salt concentrations (50 mM and 100 mM). The 50 mM elution fraction is the fraction of interest, since the rTbpB protein therein is the purest and is present in a greater amount (2.6 times more protein than in the 100 mM NaCl fraction).

The pH of the fraction corresponding to the 50 mM NaCl elution peak is decreased, with magnetic stirring, to pH 5.5 by adding 1.7N acetic acid. The solution (860 ml) is dialyzed against 5 liters of 10 mM sodium acetate buffer containing 1M urea and 0.2% elugent, pH 5.5 (24 hours at +4° C.) and then against 4 liters of 10 mM sodium acetate buffer containing 1M urea and 0.2% elugent, pH 5.5 (17 hours at +4° C.).

### Purification by Cation Exchange Chromatography on SP Sepharose (SPI) at pH 5.5

A K50 column or 100 ml of new SP Sepharose Fast Flow gel (Ge Healthcare, ref. 17-0729-01) is equilibrated in 10 mM sodium acetate buffer containing 1M urea and 0.2% elugent, pH 5.5.

The dialyzed protein solution (850 ml) is loaded on the column (flow rate 6 ml/minute). Then, five saline elution steps are carried out: 50 mM, 100 mM, 250 mM, 500 mM and 1M NaCl, in the buffer mentioned above.

The rTbpB protein is eluted exclusively in the 250 mM NaCl fraction and the low-molecular-weight contaminants are eliminated essentially in the direct eluate (40%). Approximately 35 mg of purified rTbpB M982 are thus obtained and a bit less as regards the rTbpB B16B6.

### Dialysis and Concentration of the SPI Product (250 Mm Fractions)

The fractions corresponding to the 250 mM elution peak of the SPI column are combined (volume 274 ml). The pH of the solution is brought back up to pH 7.3 by adding, with stirring, approximately 800 μl of 0.5N NaOH. The solution is dialyzed at +4° C. (Spectra Por 1: cutoff threshold 6-8000 D) against two 10 liter baths of PBS containing 0.2% elugent, pH 7.1 (66 hours and 22 hours).

The dialyzate is concentrated to a volume of 21.1 ml by frontal diafiltration concentration on a 30 kD Amicon membrane in PBS (ref. PBTK06510).

The concentrate obtained is then again dialyzed against 2 liters of PBS containing 0.2% elugent, pH 7.1 (Slide A Lyser ref. 66810: cutoff threshold 10 kD).

The solution is then filtered aseptically through a 0.22 μm Millex filter with Durapore membrane (Millipore ref SLGV 033RS). The purified rTbpB protein batch obtained is frozen at −80° C. The protein concentration is 1642 μg/ml.

1.3. Preparation of rTbpB for Injection

The rTbpB solution obtained in section B.1.2. is treated by adsorption on Bio-Beads™ SM-2 in order to remove the excess Elugent™ detergent (surfactant constituted of alkyl glucosides) which could destabilize the LOS liposomes.

### Activation of Bio-Beads™

Approximately 2.5 ml of methanol are added to 500 mg of Bio-Beads™ and the mixture is homogenized intermittently for 15 min at ambient temperature. After a settling-out period, the supernatant is removed. This washing operation is repeated twice.

Approximately 5 ml of ultrafiltered sterile water are then added and the mixture is homogenized intermittently for 15 min at ambient temperature. After a settling-out period, the supernatant is removed. This washing operation is repeated twice.

Approximately 5 ml of PBS are then added and the mixture is homogenized intermittently for 15 min at ambient temperature. It is stored at 5° C. and used the same day.

At the end, the weight of the Bio-Beads™ has increased by a factor R (equal to approximately 1.2).

### Removal of the Detergent by Adsorption on Bio-Beads™

The rTbpB solution obtained in section 1.2. contains 2 mg/ml of Elugent™. The amount of Bio-Beads™ that has to be used is determined according to the amount of Elugent™ to be removed.

For one ml of the rTbpB solution obtained in section B.1.2., 29×R mg of activated Bio-Beads™ are added. The mixture is vigorously stirred for one hour at ambient temperature. The maximum amount of liquid is then recovered and a final concentration of 0.001% of merthiolate is added thereto. The whole process is carried out under sterile conditions.

### 2. Preparation of the Purified LOS

### Culturing

Eight ml of frozen sample of the N. meningitidis C708, serogroup A, lpt3 FL lpt6 TR lgtA:: erm strain obtained above in A.4.6. or of the N. meningitidis serogroup A strain A1 known to exclusively express LOS immunotype L8 and the LOS of which bears 2 PEAs, one in position 3, the other in position 6 of the heptose II, are used to inoculate 800 ml of Mueller-Hinton medium (Merck) supplemented with 4 ml of a solution of glucose at 500 g/l and divided up in Erlenmeyer flasks. The culturing is continued with shaking at 36±1° C. for approximately 10 hours.

400 ml of a solution of glucose at 500 g/l and 800 ml of a solution of amino acids are added to the preculture. This preparation is used to inoculate a fermentor containing Mueller-Hinton medium, at an OD₆₀₀ₙₘ close to 0.05. The fermentation is continued at 36° C., at pH 6.8, 100 rpm, pO₂ 30% under an initial airstream of 0.75 1/min/1 of culture.

After approximately 7 hours (OD₆₀₀ₙₘ of approximately 3), Mueller-Hinton medium is added at a rate of 440 g/h. When the glucose concentration is less than 5 g/l, the fermentation is stopped. The final OD₆₀₀ₙₘ is commonly between 20 and 40. The cells are harvested by centrifugation and the pellets are frozen at −35° C.

Purification (Method Adapted by Westphal & Jann, (1965) Meth. Carbohydr. Chem. 5: 83)

The pellets are thawed and suspended with 3 volumes of 4.5% (vol./vol.) phenol with vigorous stirring for 4 hours at approximately 5° C. The LOS is extracted by phenol treatment.

The bacterial suspension is heated to 65° C. and then mixed vol./vol. with 90% phenol, with vigorous stirring for 50-70 min at 65° C. The suspension is subsequently cooled to ambient temperature and then centrifuged for 20 min at 11 000 g. The aqueous phase is removed and stored, while the phenolic phase and the interphase are harvested so as to be subjected to a second extraction.

The phenolic phase and the interphase are heated to 65° C. and then mixed with a volume of water equivalent to that of the aqueous phase previously removed, with vigorous stirring for 50-70 min at 65° C. The suspension is subsequently cooled to ambient temperature and then centrifuged for 20 min at 11 000 g. The aqueous phase is removed and stored, while the phenolic phase and the interphase are harvested so as to be subjected to a third extraction identical to the second.

The three aqueous phases are dialyzed separately, each against 40 1 of water. The dialyzates are then combined. One volume of 20 mM Tris, 2 mM MgCl₂ is added to 9 volumes of dialyzate. The pH is adjusted to 8.0±0.2 with 4N sodium hydroxide.

Two hundred and fifty international units of DNAse are added per gram of pellet. The pH is adjusted to 6.8±0.2. The preparation is placed at 37° C. for approximately 2 hours with magnetic stirring, and then subjected to filtration through a 0.22 μm membrane. The filtrate is purified by passing it through a Sephacryl S-300 column (5.0×90 cm; Pharmacia™).

The fractions containing the LOS are combined and the MgCl₂ concentration is increased to 0.5M by adding powdered MgCl₂.6H₂O, with stirring.

While continuing the stirring, dehydrated absolute alcohol is added to give a final concentration of 55% (vol./vol.). The stirring is continued overnight at 5±2° C., and then centrifugation is carried out at 5000 g for 30 min at 5±2° C. The pellets are resuspended with at least 100 ml of 0.5M MgCl₂ and then subjected to a second alcoholic precipitation identical to the preceding one. The pellets are resuspended with at least 100 ml of 0.5M MgCl₂.

The suspension is subjected to a gel filtration as previously described. The fractions containing the LOS are combined and filtration-sterilized (0.8-0.22 μm) and stored at 5 ±2° C.

This purification method makes it possible to obtain approximately 150 mg of LOS per liter of culture.

### 3. Preparation of [LOS] Liposomes by Detergent Dialysis

### 3.1. Preparation of Liposomes

The LOS liposomes are prepared by detergent dialysis. Briefly, the lipids (EDOPC:DOPE) are made into the form of a lipid film and taken up in 10 mM Tris buffer, and then dispersed in the presence of 100 mM of octyl-β-D-glucopyranoside (OG) (Sigma-Aldrich ref. O8001) and filtered sterilely. The LOS in 100 mM OG is added sterilely. The lipids/LOS/OG mixture is then dialyzed against 10 mM Tris buffer in order to remove the OG and to form the liposomes.

### Protocol

A lipid preparation in chloroform, of the lipids that will be used to produce the liposomes, is prepared. A dry film is obtained by complete evaporation of the chloroform.

A dry film of 1,2-dioleoyl-sn-glycero-3-ethylphosphocholine (EDOPC or ethyl-DOPC) and of 1,2-dioleoyl-sn-glycero-3-phosphoethanolamine (DOPE) in an EDOPC:DOPE mole ratio of 3 to 2 is obtained by mixing 12.633 ml of a solution of EDOPC (Avanti Polar Lipids ref. 890704) at 20 mg/ml in chloroform and 7.367 ml of a solution of DOPE (Avanti Polar Lipids ref. 850725) at 20 mg/ml in chloroform, and evaporating off the chloroform until it has completely disappeared.

The dry film is taken up with 30 ml of 10 mM Tris buffer, pH 7.0, so as to obtain a suspension containing 13.333 mg of lipids/ml (8.42 mg/ml of EDOPC and 4.91 mg/ml of DOPE). The suspension is stirred for 1 hour at ambient temperature and then sonicated for 5 min in a bath.

3.333 ml of a sterile 1M solution of octyl-[3-D-glucopyranoside (OG) (Sigma-Aldrich ref. O8001) in 10 mM Tris buffer, pH 7.0, are then added, still with stirring, so as to obtain a clear suspension of lipids at 12 mg/ml, 100 mM OG and 10 mM Tris buffer. The stirring is continued for 1 h at ambient temperature on a platform shaker. Filtration is then carried out sterilely through a Millex HV 0.45 μm filter.

A composition is prepared, under sterile conditions, by bringing together LOS and lipids in a lipids:LOS mole ratio of 250 (0.160 mg/ml of LOS, 9.412 mg/ml of lipids and 100 mM of OG). 40 ml of such a composition are obtained from mixing the following preparations:

2.005 ml of 10 mM Tris buffer, pH 7.0; 0.223 ml of 100 mM OG in 10 mM Tris; 31.373 ml of the EDOPC:DOPE suspension having a mole ratio of 3:2, at 12 mg/ml in 100 mM OG, 10 mM Tris; and 6.4 ml of a sterile suspension of LOS at 1 mg/ml in 100 mM OG, 10 mM Tris.

After stirring for one hour at ambient temperature, the suspension is transferred sterilely into 4 sterile 10 ml dialysis cassettes. Each cassette is dialyzed 3 times (24 hrs-24 hrs-72 hrs) against 200 volumes of 10 mM Tris, pH 7.0, i.e. 2 1.

The liposomes are recovered under sterile conditions. The increase in volume after dialysis is approximately 30%.

Merthiolate and NaCl are added to this preparation so as to obtain a preparation of liposomes in 10 mM Tris, 150 mM NaCl, pH 7.0, 0.001% merthiolate, which ultimately contains approximately 110 μg/ml of LOS and 7 mg/ml of lipids, of which there are approximately 4.5 mg/ml of EDOPC and approximately 2.5 mg/ml of DOPE (theoretical concentrations).

The LOS liposomes are stored at +5° C.

### 3.2. Preparation of the Injectable Materials

The liposomes are adjusted to the required LOS concentration (in particular required for the immunogenicity tests) in 10 Mm Tris, 150 Mm NaCl, pH 7.4. The merthiolate concentration is maintained at 0.001%.

4. Preparation of an [LOS] Liposomes +rTbpB Mixture

rTbpB in PBS (section B.1.3.) is mixed with [LOS] liposomes (section B.3.) in an rTbpB:LOS weight:weight ratio equal to 1. The volume is then adjusted with 10 mM Tris buffer containing 150 mM NaCl, pH 7.4, so as to obtain a preparation in which each of the components (rTbpB and LOS) is at a concentration of 80 μg/ml. The merthiolate concentration is maintained at 0.001%.

### 5. Immunogenicity Study in Rabbits

The various formulations tested were produced as described in one of the preceding sections.

### 5.1. Immunization of the Rabbits

24 7-week-old NZ KBL rabbits (Charles River Lab.) were divided into 4 test groups of four (groups A to D) and into 4 groups of two (groups E to H).

The rabbits of each group receive in a volume of 0.5 ml divided into 2 concomitant intramuscular injections into the legs, on D0, D21 and D42:
Group A: 40 μg of liposomes [LOS a chain L8, PEA-3, PEA-6] and 40 μg rTbpB M982,
   in Tris 10 mM NaCl 150 mM pH 7.4 buffer;
Group B: 40 μg of liposomes [LOS α chain L8, PEA-3, PEA-6] and 40 μg rTbpB B16B6,
   in Tris 10 mM NaCl 150 mM pH 7.4 buffer;
Group C: 40 μg of liposomes [LOS α chain L8, PEA-3] and 40 μg rTbpB M982,
   in Tris 10 mM NaCl 150 mM pH 7.4 buffer;
Group D: 40 μg of liposomes [LOS a chain L8, PEA-3, PEA-6] in Tris 10 mM NaCl 150 mM pH 7.4 buffer;
Group E: 40 μg rTbpB M982 and 40 μg of LOS-free liposomes in Tris 10 mM NaCl 150 mM, Tween 0.5% pH 7.0 buffer;
Group F: 40 μg rTbpB B16B6 and 40 μg of LOS-free liposomes in Tris 10 mM NaCl 150 mM, Tween 0.5% pH 7.0 buffer;
Group G: 40 μg of liposomes [LOS α chain L8, PEA-3] in Tris 10 mM NaCl 150 mM pH 7.4 buffer;
Group H: Tris 10 mM NaCl 150 mM pH 7.4 buffer

Animal blood is collected for analysis on D0, D42 (before the third injection) and on D56.

5.2. Measurement of the Bactericidal Activity of the Purified IgGs Against Strains of N. meningitidis Heterologous to the Strain C708

Starting with the serum pools, the IgGs were purified by affinity chromatography using the HiTrap rProtein A FF column (GE Healthcare/Amersham Biosciences) according to the manufacturer's recommendations.

Using the purified IgGs, serial two fold dilutions were prepared in gelatinized Dulbecco's PBS containing calcium and magnesium ions. The dilutions are prepared in a 96-well plate for a final volume of 50 μl per well.

The bactericidal activity of the purified IgGs was tested against the strains cited in Table II below.

A culture of the strains of N. meningitidis is prepared in supplemented or unsupplemented BHI medium for 2 hours 30 minutes with Desferal 50 μM (iron chelating agent in free form, which allows the expression of TbpB).

25 μL of the culture in exponential phase (4×10³ CFU/mL) and 25 μL of baby rabbit complement at 1/1.5 are added to each well. The plate is incubated for one hour at 37° C., with agitation.

50 μL of the mixture of each well are then deposited onto bioMérieux Mueller-Hinton agar dishes and incubated overnight at 37° C. under 10% CO₂. The number of clones is counted.

There are three controls:

Bacteria+baby rabbit complement, without serum to be tested (“complement” control);

Bacteria+inactivated baby rabbit complement, without serum to be tested (“microorganisms” control); and

Bacteria+inactivated baby rabbit complement+serum to be tested (serum control).

The bactericidal titer is expressed as being the inverse of the dilution giving 50% bacterial death by comparison with the “complement” control.

### 5.3. Results and Discussion

### Bactericidal Test

34 strains of N. meningitidis were cross-tested for their bactericidal effect. Their names are given in Table II below.

The bactericidal activity of the purified IgGs is expressed in “fold increase”. The “fold increase” is the ratio of the bactericidal titer of the purified IgGs of the group of interest: bactericidal titer of the corresponding negative control group. Thus, the degree of seroconversion in “fold increase” measures the increase of the bactericidal titer. It is considered that the bactericidal activity is significant when a factor ×8 is observed between the group of interest and the corresponding negative control group (“fold increase” greater than or equal to ×8).

As expected, the purified IgGs from the negative control immunization group do not show any bactericidal activity against any of the strains (“fold increase” greater than ×4).

The purified IgGs obtained from the immunization groups D and G (no addition of LOS with a TbpB) show a reduced level of crossed bactericidal effect. Table II below presents only the bactericidal effect results expressed in “fold increase” obtained with the purified IgGs of groups A, B, C, E and F.

Table III presents the bactericidal effect results expressed in “fold increase”, for the purified IgGs obtained from group A towards 22 strains cultured in the presence/absence of Desferal.

Table IV presents, for various vaccine compositions, the percentage of protection deduced from the crossed bactericidal effect study including 34 strains cultured in the presence of Desferal.

TABLE II

| 34 strains included in | | | LOS in liposomes: | | | | |
|---|---|---|---|---|---|---|---|
| the crossed bactericidal | | | L8 PEA-3, -6 + | | L8 PEA-3, -6 + | Empty liposomes: + | |
| effect study, cultured | | | lipidized | L8 PEA-3 + | lipidized | Lipidized | |
| in the presence of Desferal | | | TbpB | lipidized | TbpB | TbpB | Lipidized |
| | TbpB | | M982 | TbpB M982 | B16B6 | M982 | TbpB B16B6 |
| IT | isotype | Name | Group A | Group C | Group B | Group E | Group F |
| L3 | II | BZ83 | x 32 | x 16 | <x 4 | x 16 | <x 4 |
| | II | LNP23015 | x 16 | x 16 | <x 4 | x 16 | <x 4 |
| | II | LNP20443 | ≦x 4 | <x 4 | <x 4 | <x 4 | <x 4 |
| | II | LNP22979 | x 32 | x 8 | <x 4 | <x 4 | <x 4 |
| | II | BZ138 | x 256 | x 256 | <x 4 | x 256 | <x 4 |
| | II | 95/46 | x 16 | x 8 | <x 4 | x 64 | <x 4 |
| | II | S3032 | x 4 | x 4 | <x 4 | <x 4 | <x 4 |
| | II | LNP22763 | x 4 | <x 4 | <x 4 | <x 4 | <x 4 |
| | II | M982 | x 512 | x 512 | <x 4 | x 256 | <x 4 |
| | II | NG144/82 | x 138 | x 64 | <x 4 | x 128 | <x 4 |
| | II | H44/76 | x 4 | x 4 | <x 4 | <x 4 | <x 4 |
| | II | MC58 | x 8 | x 4 | <x 4 | x 16 | <x 4 |
| | II | NGPB24 | x 4 | x 8 | <x 4 | x 16 | <x 4 |
| | II | NGF26 | x 4 | <x 4 | <x 4 | <x 4 | <x 4 |
| L4- | II | BZ163 | x 16 | x 8 | x 4 | <x 4 | <x 4 |
| like | I | NGP20 | <x 4 | <x 4 | x 1024 | <x 4 | x 1024 |
| | I | M986 | <x 4 | <x 4 | x 1024 | <x 4 | x 512 |
| L4 | II | M2 | <x 4 | <x 4 | <x 4 | <x 4 | <x 4 |
| L8 | II | 8680 | x 256 | x 256 | x 16 | <x 4 | <x 4 |
| | II | RH873 | x 256 | x 256 | x 64 | x 8 | <x 4 |
| L1 | II | 92/123 | x 32 | x 32 | x 16 | x 4 | <x 4 |
| | II | M101/93 | x 16 | x 16 | <x 4 | x 4 | <x 4 |
| | II | 1000 | x 4 | x 4 | x 8 | <x 4 | <x 4 |
| | I | No. 28 LO | x 8 | x 16 | x 128 | x 8 | x 16 |
| | | 05-2606 | | | | | |
| | II | No. 60 AA | x 128 | x 256 | x 64 | x 32 | <x 4 |
| | | 07-1734 | | | | | |
| L2 | II | BZ157 | <x 4 | <x 4 | <x 4 | <x 4 | <x 4 |
| | II | BZ232 | x 32 | x 16 | <x 4 | x 16 | <x 4 |
| | I | B16B6 | <x 4 | <x 4 | x 1024 | <x 4 | x 512 |
| N.d. | II | 30 | x 128 | x 64 | <x 4 | x 16 | <x 4 |
| | II | 62 | x 8 | x 4 | <x 4 | <x 4 | <x 4 |
| | I | FAM18 | <x 4 | <x 4 | x 1024 | <x 4 | x 512 |
| | II | 90/94 | <x 4 | x 4 | <x 4 | x 4 | <x 4 |
| | II | 22 | x 16 | x 32 | x 16 | <x 4 | <x 4 |
| | II | EG327 | <x 4 | <x 4 | <x 4 | <x 4 | <x 4 |

In Table II above:
IT means “immunotype”
L6 means an LOS bearing an α chain of L6 type
L8 means an LOS bearing an α chain of L8 type
PEA-3 means that the LOS bears only one PEA substituent in position 3 of the heptose II
PEA-3, -6 means that the LOS bears a PEA substituent in position 3 and a PEA substituent in position 6 of the heptose II.

TABLE III

| LOS in liposomes: | | | | L8 PEA-3, -6 + | |
|---|---|---|---|---|---|
| Strains included in the crossed bactericidal | | | | lipidized | |
| effect study, cultured in the presence | | | | TbpB M982 | |
| (Desferal)/absence of chelating agent | | | | Group A | |
| | | Epidemio- | | Without | With |
| | TbpB | logical | | chelating | chelating |
| IT | isotype | complex | Name | agent | agent |
| L3 | II | ST-32 | BZ83 | < x 4 | x 32 |
| | II | ST-41/44 | LNP23015 | < x 4 | x 16 |
| | II | ST-41/44 | LNP22979 | < x 4 | x 32 |
| | II | ST-41/44 | BZ138 | x 32 | x 256 |
| | II | ST-41/44 | 95/46 | < x 4 | x 16 |
| | II | | S3032 | < x 4 | x 4 |
| | II | | LNP22763 | < x 4 | x 4 |
| | II | | M982 | x 64 | x 512 |
| | II | | NG144/82 | x 4 | x 128 |
| | II | | H44/76 | < x 4 | x 4 |
| | II | | NGF26 | < x 4 | x 4 |
| | II | | 62 | < x 4 | x 8 |
| L4- | II | ST-8 | BZ163 | x 8 | x 16 |
| like | | | | | |
| L8 | II | | 8680 | x 128 | x 256 |
| | II | ST-41/44 | RH873 | x 64 | x 256 |
| L1 | II | ST-41/44 | 92/123 | x 16 | x 32 |
| | I | ST-269 | No. 28 LO | < x 4 | x 8 |
| | | | 05-2606 | | |
| | II | ST-269 | No. 60 AA | x 64 | x 128 |
| | | | 07-1734 | | |
| N.d. | II | | EG327 | < x 4 | < x 4 |
| L2 | II | | BZ157 | < x 4 | < x 4 |
| | II | | BZ232 | < x 4 | x 32 |
| | I | | B16B6 | < x 4 | < x 4 |

TABLE IV

| | % of protection deduced from |
|---|---|
| | the crossed bactericidal effect |
| | studies including 34 strains |
| | cultured in the presence of |
| Vaccine compositions | Desferal |
| L8 PEA O3, O6 + TbpB M982 | 55.9% |
| L8 PEA O3 + TbpB M982 | 52.9% |
| L8 PEA O3, O6 + TbpB B16B6 | 32% |
| L8 PEA O3, O6 + TbpB M982 + | 58.8-67.6% |
| TbpB B16B6 | |
| L8 PEA O3 + TbpB M982 + | 64.7% |
| TbpB B16B6 | |

1. A vaccine for combating N. meningitidis infections, which comprises:
(iii) an LOS of N. meningitidis formed especially from a lipid A, an inner core, an α chain of L8 type, in which the heptose II residue of the inner core (a) bears in position O-3 a phosphoethanolamine (PEA) substituent and does not bear a PEA substituent in positions O-6 and O-7; or (b) bears a phosphoethanolamine (PEA) substituent in position O-3 and in position O-6 or O-7; and
(iv) the lipidized subunit B (Tpb B) of the human transferrin receptor of a strain of N. meningitidis or a lipidized fragment of this TpbB.

2. The vaccine as claimed in claim 1, which comprises:
(iv) an LOS of N. meningitidis formed especially from a lipid A, an inner core, an α chain of L8 type, in which the heptose II residue of the inner core bears in position O-3 a phosphoethanolamine (PEA) substituent and does not bear a PEA substituent in positions O-6 and O-7;
(v) an LOS of N. meningitidis formed especially from a lipid A, an inner core, an α chain of L8 type, in which the heptose II residue of the inner core bears a phosphoethanolamine (PEA) substituent in position O-3 and in position O-6 or O-7; and
(vi) the TpbB of N. meningitidis or a lipidized fragment of the latter.

3. The vaccine as claimed in claim 1, in which the TbpB is the TbpB of a strain of N. meningitidis of isotype II.

4. The vaccine as claimed in claim 3, in which the TbpB of a strain of N. meningitidis of isotype II is the TbpB of the strain of N. meningitidis M982.

5. The vaccine as claimed in claim 3, which also comprises the TbpB of a strain of N. meningitidis of isotype I.

6. The vaccine as claimed in claim 5, in which the TbpB of the strain of N. meningitidis of isotype I is the TbpB of the strain of N. meningitidis B16B6.

7. The vaccine as claimed in claim 1, in which the LOS is formulated as liposomes (LOS liposomes).

8. The vaccine as claimed in claim 1, in which the LOS and the TbpB(s) are formulated together in liposomes.

9. The vaccine as claimed in claim 1, in which the LOS is formulated as liposomes and in which the TbpB(s) are mixed with the LOS liposomes.

10. The vaccine as claimed in claim 1, which does not contain any outer membrane vesicle (OMV) of N. meningitidis.
